(19) United States
(12) Patent Application Publication
(10) Pub. No.: US 2014/0364454 A1
(43) Pub. Date: Dec. 11, 2014

(54) METAL-CHELATING COMPOUNDS HAVING AT LEAST ONE POLYAMINO CHAIN

(71) Applicants: UNIVERSITE DE RENNES I, RENNES Cedex (FR); UNIVERSITE DE NANTES, NANTES (FR); CNRS, PARIS Cedex 16 (FR)

(72) Inventors: Francois Gaboriau, Rennes (FR); David Deniaud, Saint Herblain (FR); Vincent Corce, Nantes (FR); Karine Julienne Aphecetche, Sainte-Luce-Sur-Loire (FR); Eric Renault, Nantes (FR)

(21) Appl. No.: 14/367,493

(22) PCT Filed: Dec. 18, 2012
(86) PCT No.: PCT/EP2012/075907

§ 371 Date: Jun. 20, 2014

(30) Foreign Application Priority Data
Dec. 20, 2011 (FR) 1162108

(51) Int. Cl. C07D 215/26; A61K 45/06; A61K 31/47

(52) U.S. Cl. CPC C07D 215/26; A61K 31/47; A61K 45/06
USPC 514/311; 546/166

(57) ABSTRACT

The invention relates to metal-chelating compounds comprising one or more 8-hydroxyquinoline units substituted in position 2 with a nitrogenous group comprising a polyamino chain, wherein the polyamino chain is N-substituted on said nitrogenous group, to the composition containing same, and to the uses thereof in the therapeutic field and in particular in the treatment of diseases associated with an abnormality in the regulation of metal metabolism resulting in a metal overload in human or animal cells.

The present invention relates to chimeric molecules associating one or more metal-chelating groups of the 8-hydroxyquinoline type with at least polyamine chain. These compounds are here below designated by the acronym “Quilamine.

The invention also relates to the use of such Quilamines in the therapeutic field, especially in the treatment or prevention of proliferative diseases and/or diseases related to metal overloads such as cancers and neurodegenerative diseases among humans and animals.

Many metal ions derived from iron, copper, manganese, zinc, come into play in several biochemical and metabolic reactions of the organism. The needs of the organism generally vary according to the type of metal ions and according to the location of the biochemical and metabolic reactions brought into play. In order to ensure efficient functioning of the organism, these metal ions must be present in determined levels to prevent overloads or deficiencies for example. In particular, overloads in metal ions such as iron and copper are harmful or even toxic for the organism because they catalyze the formation of reactive species of oxygen. Overloads in metal ions can have various causes: genetic, metabolic, exogenous nutritional inputs or repeated blood transfusions. In particular, they can be related to diseases such as cancers or tumors, neurodegenerative diseases in which there is an imbalance in the homeostasis of the metal ions.

In the case of cancers, the tumor cells proliferate uncontrollably and require abnormally high quantities of metal ions such as iron and copper for their growth, as compared with healthy cells.

The chelators initially proposed for the treatment of primary ion overloads such as Desferal®, and those more recently developed for the treatment of secondary overloads (thalassemia), such as Deferiprone® and Deferasirox®, inhibit the proliferation of hepatic tumor cells of rats in culture (Richardson, D. R., Potential of iron chelators as effective anti-proliferative agents. Can J Physiol Pharmacol, 1997. 75(10-11): p. 1164-80). It has been shown that the anti-proliferative activity of Quilamine HQ1-44 is greater than that of Deferasirox® in human tumor cells of hepatic origin and of enterocytic origin. Desferal® inhibits in vitro and in vivo growth of melanomia cells (Richardson, D., P. Ponka, and E. Baker, The effect of the iron(III) chelator, desferrioxamine, on iron and transferrin uptake by the human malignant melanoma cell. Cancer Res, 1994. 54(3): p. 685-9) and of hematomas (Yamasaki, T., S. Terai and I. Sakaida, Deferoxamine for advanced hepatocellular carcinoma. The New England Journal of Medicine, 2011, 365(6): p. 576-77). During preliminary clinical trials these chelators proved to be efficient in the treatment of leukemias and neuroblastomas. Besides, the mode of action of a cytotoxic chemotherapy agents such as doxorubicin and bleomycin partially bring their iron and copper chelating capacity into action, in exactly the same way as clioquinol, derived from 8-hydroxyquinoline (Ding, W. Q., and al., Anticancer activity of the antibiotic clioquinol. Cancer Res, 2005. 65(8): p. 3389-95).

Conversely, the addition of exogenous iron stimulates the proliferation of the tumor cells. The need for iron during the cellular cycle is illustrated by the hyper-expression on the plasma membrane of the proliferating cells of the transferrin receptor which enables the entry of iron bound to transferrine in the cell. Ferroportin, which is an iron-exporting protein plays a key role in the systemic regulation of this metal. It is a diagnostic marker for breast cancer. The increase of in the expression of iron-import proteins (DCYTB, DMT1 and TfR1) and the diminishing of the expression of iron-export proteins (HEPH and FPN), which lead to an increase in the intra-cell iron have been described during the progress of colorectal cancer.

In addition, the implication of the metabolism of polyamines in cell replication and therefore in the proliferative processes cause this metabolism to be one of the preferred targets of anti-proliferative drugs and, at the same time, the source of new circulating signals liable to prove the existence of a neoplasic process within the organism.

The polyamines which are found not only within the very interior of the cells but also in a state where they circulate in the biological liquids of the organism such as blood comr from three main sources:
physiological cell proliferation (growth and/or renewal of the constituent cells of the organism) and tumor proliferation,
food,
intestinal bacteria.

Biosynthesis and the picking up of these ubiquitous molecules by the polyamine transport system (PTS) is strongly activated in tumor cells. The anti-proliferative activity of iron chelators such as Deferasirox® and O-trensox is associated with intra-cell iron depletion which they provoke and the inhibition of the metabolism of polyamines (Gaboriau, F., and al., Modulation of cell proliferation and polyamine metabolism in rat liver cell cultures by the iron chelator O-trensox. Biometals, 2006. 19(6): p. 623-32).

The reactive oxygen species produced in an iron overload or copper overload situation at the cerebral level bring about the deterioration of the membrane lipids, proteins and DNA of the nerve cells which a play a role in the etiology of neurodegenerative disorders such as Alzheimer's and Parkinson's diseases. High iron concentrations are observed in the central nervous system (CNS). Independently of their implication in catalysis of the reactive oxygen species, metals such as iron and copper also seem to play a major role in the aggregation of the proteins. They are therefore liable to provide a link between the two pathological processes of protein aggregation and oxidative damage characterizing the neurodegenerative diseases, such as Parkinson's disease (PD) and Alzheimer's disease (AD) and prion diseases. Among the iron chelators tested for their efficiency in inhibiting neurodegeneration, Clioquinol which is currently the subject of clinical trials has the best protective efficacy in animal models of AD and PD.

The development of chelators of these metal ions, especially iron or copper, is a promising avenue of research in the treatment of proliferative diseases and/or diseases related to metal overload in the organisms such as cancers or certain neurodegenerative diseases. A search is on for chelators capable of inducing cellular metal depletion great enough to create intra-cell metal deficiency and reduce cell proliferation and the local production of reactive oxygen species.

However, one of the problems of these chelators is that they are taken up and complexate metal ions equally well in healthy cells and diseased cells. Even if the needs in iron and copper of the tumor cells are higher than those of normal cells, it is preferable to vectorize the chelator towards the tumor cells in order to prevent any excessively great toxicity of the treatment.

Studies have focused on the role of polyamines as intra-cell vectors of iron chelators. Natural polyamines such as putrescine, spermine, spermidine, norspermine and norspermidine, in the same way as iron, copper, manganese and zinc, are indispensable for cell growth. Polyamine deficiency caused by treatment a polyamine synthesis inhibitor, decontamination of the intestinal tract by an antibiotic and a polyamine-depleted diet slows down the growth of tumors among animals (Seiler N. Thirty years of polyamine-related approaches to cancer therapy. Retrospect and prospect. Part 1. Selective enzyme inhibitors. Current drug targets. 2003, 4:p. 537-64).

Cancer cells have a particularly amplified metabolism (biosynthesis and capture) of polyamines. The polyamine transport system (PTS) which carries out the transport of polyamines is particularly overactive in these cells. Since the recognition and transport of polyamines is not specific to natural polyamines, it has been possible to develop analogues of natural polyamines that can be recognized and transported by the PTS.

In the patent application EP 1 667 727, there are known ways of using benzylmaltol type chelators (Deferiprone® or CP20) or poly(hydroxamic acid) coupled to a polyamine chain in the treatment of cancer or of a tumor and the treatment of a patient suffering from a metal overload condition.

However, these chelators have low affinity for iron and do not give full satisfaction in the development of a therapeutic treatment, for example in the treatment of cancers. In particular, the anti-proliferative action of these chelators on a hepatic cells in culture (HuH7 cell line) is of low efficacy.

The Applicant has now discovered a novel family of compounds associating metal chelators or polyamines called Quilamines. These compounds exert anti-proliferative action efficaciously and selectively on diseased cells and remedy the above-mentioned drawbacks. The Quilamines according to the invention are compounds comprising at least one 8-hydroxyquinoline motif substituted in position 2, i.e. in an alpha position of the nitrogen atom forming the 8-hydroxyquinoline cycle, by at least one polyamine chain. Through their particular structure, the Quilamines according to the invention are capable of providing treatment for proliferative diseases and/or diseases linked to metal overload in human and animal cells, such as cancers or neurodegenerative diseases that is more targeted, less toxic and more efficacious than that provided with known chelators.

In particular, it has been observed that, on a same type of cell line, a Quilamine according to the invention had high anti-proliferative activity that can be even greater than that of classic iron chelators for a same concentration of chelators. The Quilamines according to the invention have anti-proliferative activity that is reinforced or at least unchanged in the presence of exogenous iron as compared with classic iron chelators. They are particularly efficacious for processing diseases related to serum iron overload in human or animal cells.

In addition, Quilamines according to the invention have the advantage of low toxicity and are appropriate for different applications in the therapeutical field. They have low cytotoxicity for healthy cells, especially because of their high selectivity of recognition by the polyamine transportation system, enabling them to selectively target tumor cells.

Another object of the present application pertains to Quilamines according to the invention as agents of therapeutic treatment. An object of the invention is also Quilamines according to the invention as agents for the treatment of proliferative diseases and/or neurodegenerative diseases related to metal overload, for example iron and/or copper overload. The Quilamines according to the invention are particularly useful as anti-proliferative agents and/or cytotoxic agents for cancer or tumor cells.

The compounds according to the invention can be obtained according to methods of preparation in which the polyamine chain or chains is (or are) coupled to 8-hydroxyquinoline motifs by steps of reductive amination, nucleophilic substitution or Michael addition to give a Quilamine according to the invention. These methods of preparation have the advantage of being simple to carry out. In particular, they enable the synthesizing of the Quilamines according to the invention in less than 15 steps, and preferably in less than 12 steps.

Other objects, aspects and features of the invention shall appear more clearly from the description and from the examples.

The invention relates to compounds comprising at least one 8-hydroxyquinoline motif comprising at least one polyamine chain in position 2, i.e. in the alpha position of the nitrogen atom constituting the 8-hydroxyquinoline cycle.

More specifically, the Quilamines according to the invention respond to the following general formula (I) or to one of its conjugate forms, its salts or its solvates:

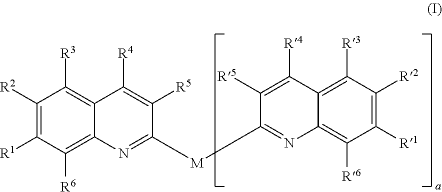

wherein:
R¹, R², R³, R⁴ and R⁵ are chosen independently of one another from among a hydrogen atom, a linear or branched, saturated and non-saturated, cyclic or aromatic hydrocarbon group comprising 1 to 10 carbon atoms, an aliphatic or aromatic cyclic hydrocarbon group comprising 4 to 12 carbon atoms, a halogen, a thiol, a hydroxyl, an amine that is preferably a secondary or tertiary amine, an ether, a thioether;
R⁶ is a hydroxyl;
a is equal to 0 or 1 with:
   when a is equal to 0, M is a group responding to the following formula (I′):

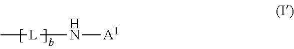

wherein:
b being equal to 0 or 1,
L representing an alkyl group having 1 to 4 carbon atoms, a group C═O or C═S,
A¹ responding to the general formula (X) or (Y):

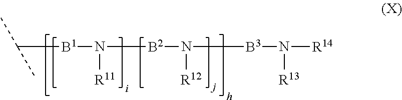

wherein:
R¹¹, R¹², R¹³ and R¹⁴ are chosen independently of one another from among hydrogen atoms, linear or branched, saturated, unsaturated, cyclic or aromatic hydrocarbon groups comprising 1 to 12 carbon atoms, and amine function protecting groups,
B¹, B² and B³ are chosen independently of one another from among linear or branched, saturated or unsaturated, cyclic or aromatic hydrocarbon groups comprising 2 to 6 carbon atoms
i and j, identical or different are equal to 0 or to 1,
h is an integer ranging from 0 to 4;

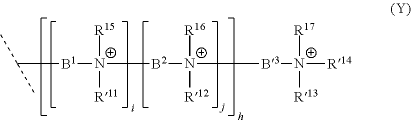

wherein:
R′¹¹, R′¹², R′¹³, R′¹⁴, R¹⁵, R¹⁶, and R¹⁷ are chosen independently of one another from among the linear or branched, saturated, unsaturated, cyclic or aromatic hydrocarbon groups comprising 1 to 12 carbon atoms and amine function protecting groups.
B^{1 and} B² are chosen independently of one another from among the linear or branched, saturated or unsaturated hydrocarbon groups comprising 2 to 6 carbon atoms,
B′³ is chosen from the linear or branched or saturated, unsaturated hydrocarbon groups comprising 2 to 6 carbon atoms capable of being interrupted by one or more secondary amine functions —NH— and/or one or more tertiary amine functions —NR¹¹—, —NR¹²—.
i and j, which are identical or different, are equal to 0 or to 1,
h is an integer ranging from 0 to 4;
and when a is equal to 1, M is a group meeting the following formula (I″):

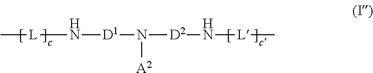

wherein:

c and c′, identical or different, being equal to 0 or to 1,

L and L′, identical or different, represent an alkyl group having to 1 to 4 carbon atoms, a group C═O or C═S,

D^{1 and} D², identical or different, represent a linear or branched hydrocarbon group, comprising 1 to 5 carbon atoms,

A² being identical or different from A¹ and meeting the formula (X) or (Y);

R′¹, R′², R′³, R′⁴, R′⁵ and R′⁶ are chosen independently of one another from among a hydrogen atom, a linear or branched, saturated or unsaturated hydrocarbon group comprising 1 to 10 carbon atoms, a cyclic, aliphatic or aromatic, possibly substituted hydrocarbon group comprising 4 to 12 carbon atoms, a halogen, a thiol, hydroxyl, a preferably secondary or tertiary amine, an ether, a thioether.

The Quilamine according to the invention is a metal chelator. It is capable of binding with a metal atom or metal ion in forming several (at least two) chelator-metal coordination bonds to form a metal complex, especially with a metal atom of the 3d series of transition metals, especially iron, copper, manganese and/or zinc. The Quilamine according to the invention is especially capable of chelating an iron ion having a degree of oxidation equal to two (Fe(II) or Fe²⁺) or three (Fe(III) or Fe³⁺), a copper ion having a degree of oxidation equal to two (Cu(II) or Cu²⁺), a manganese ion having a degree of oxidation equal to two (Mn(II) or Mn²⁺) or a zinc ion having a degree of oxidation equal to two (Zn(II) or Zn²⁺). Preferably, Quilamine is an iron chelator.

The complexation affinity of Quilamine with the metal ion in the organism reflects the chelating capacity or chelating power of Quilamine. This affinity can be assessed by measuring the value of pW corresponding to −log of the concentration in free metal ion W, i.e. non-complexated by the Quilamine for a ratio of respective concentrations of Quilamine (10 micromoles per liter) and metal ion (1 micromole per liter) equal to 10, at 25° C. and pH 7.4. The higher the value of pW, the greater the affinity of Quilamine with metal ion. The affinity of Quilamine with iron in its ferric form (Fe(III)) and its ferrous form (Fe(II)) is measured respectively by pFe³⁺ and pFe²⁺ at physiological pH (pH=7.4), at 25° C. Under these conditions of measurement, the Quilamine according to the invention presents, at physiological pH, a value pFe²⁺ and/or pFe²⁺ strictly greater than that measured in identical conditions for ligands of low molecular weight usually present in animal or human cells complexating the labil iron pool or LIP. These ligands of low molecular weight usually present in cells can for example be albumen, citrate (pFe³⁺/citrate=19.3 at pH=7.4), or ascorbate.

In particular, pFe²⁺ or pFe³⁺ of the Quilamine-iron complex is strictly greater than 20 and preferably ranges from 25 to 27 physiological pH.

At physiological pH, the Quilamine forms a Quilamine-Fe(III) complex preferably having a ligand:metal stoichiometry of 2/1. The ligand:metal stoichiometry can for example be determined by the continuous variation method, using the characteristic transition in the absorption spectrum of the Quilamine-Fe(III) complex at 580 nm, for chelator and iron concentrations ranging from 0 to 500 micromoles per liter.

The polyamine chain or chains of Quilamine are recognized by the cell polyamine transport system and enables the Quilamine to be conveyed towards the cells.

Preferably, the polyamine chain or chains of Quilamine according to the invention are cationic or cationizable.

The term “cationic” is understood to mean comprising one or more quaternary amine functions such that overall ion load carried by the amine chain is positive. In this sense, polyamine chain can be monocationic or polycationic.

The term “cationizable” is understood to mean comprising one or more primary and/or secondary amine functions, especially in the form of salt, that gets ionized spontaneously into cation(s) in the medium of use of Quilamine, at physiological pH.

The polyamine chain or chains can comprise a putrescine, spermine, spermidine motif, or a non-natural analogue of these compounds such as homospermine, homospermidine, norspermine.

Advantageously, the polyamine chain or chains comprise a motif non-naturally analogue with putrescine, spermine or spermidine, such as homospermine, homospermidine, norspermine, enabling them to be recognized by the polyamine transport system but without being degraded by the enzymes undergoing oxidative retroconversion.

In a first more preferred embodiment, a is equal to zero and b is equal to one.

In a second particularly preferred embodiment, a is equal to zero, b is equal to one and L represents a CH₂ group.

In a third particularly preferred embodiment, a is equal to zero, b is equal to one and L represents a CH₂—CH₂ group.

In a fourth particularly preferred embodiment, a is equal to zero, b is equal to one and L represents a group C═O.

In a fifth particularly preferred embodiment, a is equal to zero, b is equal to one and L represents a group C═S.

In a sixth more preferred embodiment, when a is equal to 1, c and c′ are identical, L and L′ are identical, D¹ and D² are identical, and/or R¹, R², R³, R⁴, R⁵ and R⁶ are identical to R′¹, R′², R′³, R′⁴, R′⁵ and R′⁶ respectively. Preferably, R′⁶ is a hydroxyl.

In a seventh embodiment, the Quilamine according to the invention responds to the general formula (I), or to one of its conjugate forms, its salts or its solvates, to the exclusion of the following compound:

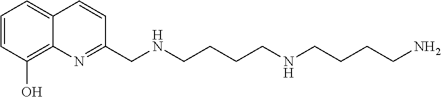

Preferably, B¹, B², B³ are chosen from the groups n-propyl and n-butyl and B′³ is chosen from the groups n-propyl and n-butyl capable of being interrupted by one or more secondary amine functions —NH— and/or one or more tertiary amine functions —NR¹¹—, —NR¹²—.

In one particular embodiment, the groups A¹ and A² mentioned further can be chosen from among:
a) those of the formula (X) in which: B¹ and B³ designate n-butyl groups, i is equal to 1, j is equal to 0, h is an even- or odd-parity integer ranging from 1 to 4, R¹³ and R¹⁴ are hydrogen atoms, R¹¹ being as defined here above; and
b) those of the formula (X) in which: B¹ and B³ designate n-propyl groups, i is equal to 1, j is equal to 0, h is an even-parity or odd-parity integer ranging from 1 to 4, R¹³ and R¹⁴ are hydrogen atoms, R^{H} being as defined here above.

In a preferred embodiment of the formulae (X) and (Y), B³ and B′³ do not designate an n-propyl group and R¹³, R¹⁴, R′¹³, R′¹⁴ and R¹⁷ do not simultaneously designate a hydrogen atom. In other words, preferably, the polyamine chain does not terminate in a primary n-propyl group. Such polyamine chains are liable to be deteriorated by amine oxidase enzymes present in serum (polyamine oxidase and semicarbazide-sensitive amine oxidase) and generate a cytotoxic aldehyde and hydrogen peroxide.

In another preferred embodiment, the Quilamine responds to the formula (I) in which a is 0, b is equal to 1, and L is a group CH₂ and A¹ or A² respond to the formula (X) in which h=i=1 and B¹ is an n-propyl group. These Quilamines have a particularly great chelating power.

Among all the Quilamines that can be used according to the invention, preference is given to those meeting the formula (I) and one of their conjugate forms, salts or solvates, in which:

R¹ to R⁵ are chosen independently of one another from among a hydrogen atom, a linear or branched, saturated or unsaturated hydrocarbon group comprising 1 to 10 carbon atoms; R⁶ is a hydroxyl; a is equal to 0; b is equal to 1; L is an alkyl group in C₁-C₂; and the group A¹ responds to the formula (X) in which R¹¹, R¹², R¹³ and R¹⁴ are chosen independently of one another from among hydrogen atoms and amine function protecting groups; B¹, B² and B³ which are identical or different, being n-propyl and/or n-butyl groups; i and j which are identical or different being equal to 1 or to 0; h being equal to 1.

In particular, the invention prefers those corresponding to the following formulae or to one of their conjugate forms, salts, or solvates:

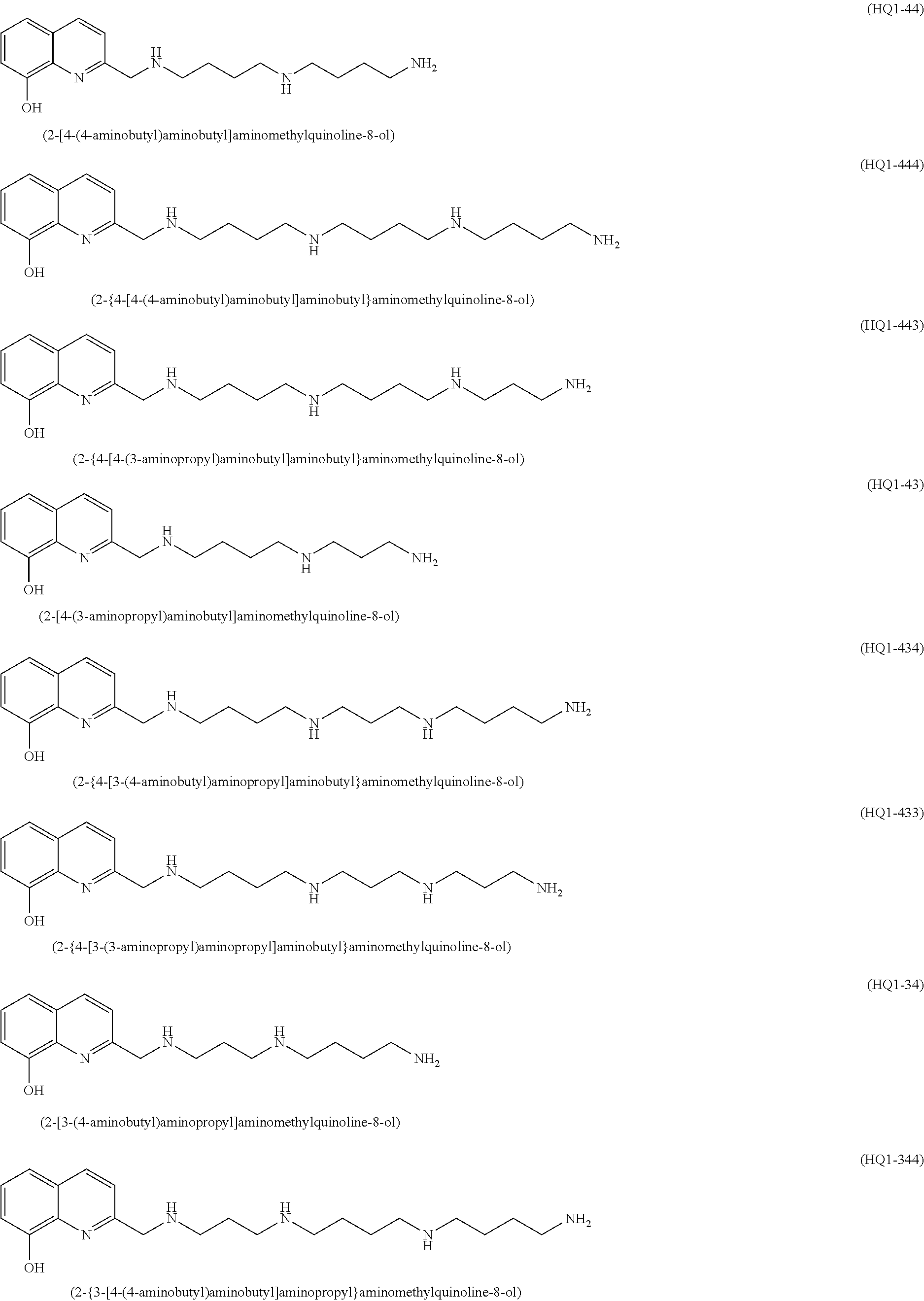

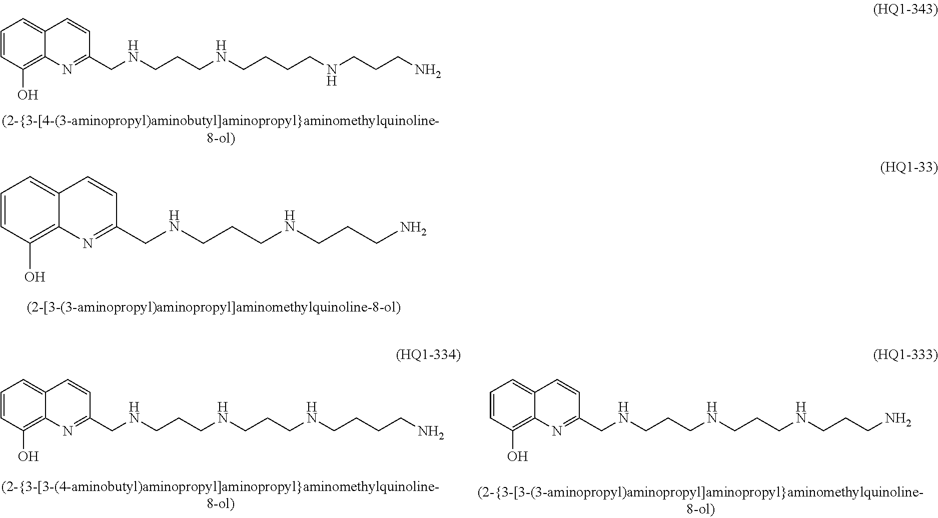 [SMILES: NCCCNCCCCNCCCNCc1ccc2cccc(O)c2n1 ; NCCCNCCCNCc1ccc2cccc(O)c2n1 ; NCCCCNCCCNCCCNCc1ccc2cccc(O)c2n1 ; NCCCNCCCNCCCNCc1ccc2cccc(O)c2n1]

Even more preferably, the invention uses the Quilamines HQ1-44, HQ1-444, HQ1-443, HQ1-43, HQ1-434, HQ1-433, HQ1-33, HQ1-334, HQ1-333, or one of their conjugate forms, salts or solvates.

The Quilamine preferred is HQ1-44 and its conjugate forms, salts or solvates. The acronym HQ1-44 describes an hydroxyquinoline HQ chelating motif indexed 1 for the group number CH₂ bound to the polyamine chain. The FIG. 44 (and respectively 444, 443, 43, 434, 433, 33, 334, 333, 34, 344, 343) specifies the number of carbon atoms between each nitrogen atom and the next of the polyamine chain.

The Quilamine salts can be chosen from among any pharmaceutically acceptable salts whatsoever. The term “pharmaceutically acceptable salt” is understood to mean especially a salt with a pharmaceutically acceptable inorganic acid, such as hydrochloric acid, sulfuric acid, phosphoric acid, nitric acid, carbonic acid, boric acid, sulfamic acid, hydrobromic acid; or else a salt with a pharmaceutically accepted organic acid such as acetic acid, propionic acid, butyric acid, tartric acid, maleic acid, hydroxymaleic acid, fumaric acid, citric acid, lactic acid, mucic acid, gluconic acid, benzoic acid, succinic acid, oxalic acid, phenylacetic acid, methanesulfonic acid, toluenesulfonic acid, benzenesulfonic acid, salicylic acid, sulfanilic acid, aspartic acid, glutamic acid, edetic acid, stearic acid, palmitic acid, oleic acid, lauric acid, panthotenic acid, tannic acid, ascorbic acid and valeric acid. When the Quilamine comprises a function that is sufficiently acid with physiological pH to react with an organic base or mineral base, the Quilamine can form a salt with said base.

The term “treatment” or “to treat” refers to the capacity of Quilamines to diminish the excess metal ion pool (iron and/or copper) and/or reduce the polyamine levels in tumor cells and consequently reduce and/or inhibit the development of these cancer or tumor cells and/or their symptoms.

The solvates of the Quilamines are constituted by complexes or aggregates formed by one or more Quilamines or one of its salts as defined here above with one or more solvent molecules. The solvents can for example be water, methanol, ethanol, isopropanol and acetic acid. When the solvent is water, the solvate is a hydrate. Preferably, the solvates of the Quilamines are hydrates such as hemihydrates, monohydrates, dihydrates, trihydrates, tetrahydrates.

The conjugate forms of the Quilamines correspond to the resonance forms due to the delocalization of one or more electronic doublets.

The Quilamine according to the invention can be used as a therapeutic agent for humans and animals, especially as agents for treating proliferative and neurodegenerative diseases and/or diseases related to an overload of iron, copper, zinc and/or manganese, in human or animal cells. In particular, Quilamine can be used as an anti-tumor agent, for example in the treatment of hepatocarcinomas, lymphomas, melanomas, renal carcinomas, ovarian carcinomas, breast cancers, cancer of the colon, and/or carcinomas of the prostate, bladder, pancreas and lungs among humans and animals. It can also be used to treat neurodegenerative diseases such as Parkinson's and Alzheimer's.

The Quilamine according to the invention can be used as an agent inhibiting the endogenous synthesis of polyamines by the organism. The polyamines are derived from the degradation of arginine, either directly by arginine decarboxylase which converts it into agmatine or via the urea cycle which begins with the conversion of arginine into ornithine, catalyzed by arginase. The polyamines are then synthesized sequentially from the ornithine which is decarboxylated initially by the decarboxylase ornithine (ODC) to form putrescine. The spermidine is synthesized by synthase spermidine which carries out the addition to the putrescine of an aminopropyl group input by S-adenosyl methionine, decarboxylated by S-adenosyl methionine decarboxylase (SAMDC). The spermine is formed in the same way out of spermidine by the addition of a second aminopropyl group. The main inhibitors of synthesis of polyamines act at the level of the ODC (α-difluoromethyl ornithine or DFMO), or the SAMDC (CGP 48664).

Another object of the invention relates to a composition comprising at least one Quilamine as defined here above.

The composition according to the invention can furthermore comprise an excipient.

The excipient or excipients that can be used are chemically inert and pharmacologically inactive auxiliary substances. In particular they have no influence on the effects of Quilamine and additional active principles, if any, present in the composition. The excipients serve to formulate the composition of the invention in the form most suited to the mode of administration desired and, possibly, if necessary, to modulate the speed of release of the active substance or substances towards the organism. As excipients for example, it is possible to cite water and saccharose which are the two excipients constituting simple syrup or again for dry forms, modified starch or starches and modified cellulose or celluloses are the disintegrating agents used in dry forms (pills, capsules, etc) to accelerate the disintegration (or again the break-up) of these medicines once they reach the stomach. For a parenteral mode of administration, the excipient can be a solvent or aqueous diluent that is sterile, isotonic relative to blood, and pharmaceutically acceptable, for example saline phosphate or saline acetate buffers, water, a 5% dextrose solution. These formulations can be prepared in the form of defined doses contained in a sterile glass flask and sealed according to the tested methods in pharmacology. The excipient is preferably a saline phosphate buffered.

The composition according to the invention can be administered by any mode of administration classically used in the therapeutic field, applied to humans or animals. In particular, it can be administered orally, sublingually, parenterally, subcutaneously, by intramuscular means, intravenously, transdermically, locally, rectally or by inhalation. Preferably, it is administered orally in a formulation using syrup, capsules or tablets.

The composition according to the invention preferably comprises a therapeutically efficacious Quilamine content, i.e. such that the composition can be used as a therapeutical agent, especially as an agent for the treatment of proliferative and neurodegenerative diseases and/or diseases related to an overload of iron, copper, zinc and/or manganese, especially as an anti-tumor agent or an agent against Parkinson's and Alzheimer's disease among humans or animals.

The composition according to the invention can furthermore comprise at least one additional active principle different from the Quilamines and having available especially a marketing authorization.

By way of additional active principles, different from Quilamines, we can cite chemotherapy agents such as alkalizing agents (cis-platin), plant alkaloids (paclitaxel, epothilones), topoisomerase inhibitors (campthotecin, taxanes, alkaloids of the Vinca family (vinblastine, vincristine, etc)), microtubule inhibitors (bleomycin) and anti-metabolites (5-Fluoro uracile) and polyamine metabolism inhibiting agents as Eflornithine (α-Difluoromethylornithine) and CGP 48664.

The composition according to the invention can furthermore include an additive chosen from among preserving agents such as methyl methylhydroxybenzoate, chlorocresol, metacresol, phenol and benzalkonium chloride.

The composition can be a ready-to-use composition or a composition obtained by an extemporaneous mixture of the Quilamine or Quilamines with one or more excipients and/or one or more additional active principles and/or one or more additives as mentioned here above.

When the composition comprises at least one Quilamine, and at least one additional active principle, the composition can be a product of combination for a use of these different active principles and Quilamines that is simultaneous, separate and spread over time.

According to one interesting variant, the additional active principle is chosen from among an agent inhibiting the endogenous synthesis of polyamines and a chemotherapeutic agent.

According to one particularly interesting variant, said agent inhibiting the endogenous synthesis of polyamines is an inhibitor of ornithine decarboxylase, S-adenosylmethionine decarboxylase, spermidine synthase or spermine synthase.

The term “agent inhibiting the synthesis of polyamines” designates a molecule capable of totally or partially, directly on indirectly, blocking at least one of the enzymes that come into play in the synthesis of polyamines in the human or animal organism. Ornithine decarboxylase (EC 4.1.1.17) is a target enzyme for compounds inhibiting the biosynthesis of polyamines. The role of the polyamine biosynthesis inhibitor to stop or significantly reduce the endogenous production of polyamines in the organism treated with the product according to the present invention. Co-treatment with such inhibitors of the endogenous synthesis of polyamines reinforces the anti-proliferative structure of polyamine according to the present invention by a combined deficiency of polyamines and intra-cell iron.

The term “chemotherapeutical agent” is understood to mean a chemical molecule used to treat diseases such as cancer, neurodegenerative diseases and autoimmune diseases. The majority of the chemotherapeutical substances work by stopping mitosis (cell division) in efficaciously targeting cells that divide far too rapidly or the synthesis and function of DNA. Certain novel agents act directly on the DNA but directly target a molecular abnormality (leukemia, cancer of the colon).

The composition according to the invention as defined here above can be used as a therapeutic agent among humans or animals, especially as an agent for treating proliferative and neurodegenerative diseases and/or diseases related to a metal overload in human or animal cells. In particular, it can be used as an agent for treating hepatic carcinomas, lymphomas, melanomas, renal, ovary, breast and colon carcinomas and/or carcinomas of the prostate, the bladder, the pancreas or the lungs among humans or animals. It can also be used to treat neurodegenerative diseases such as Parkinson's disease and Alzheimer's disease.

The compounds according to the invention can be obtained by a method in which the polyamine chain or chains are coupled to 8-hydroxyquinoline motifs by a step of reductive amination, nucleophilic substitution or Michael addition.

In particular, the method used comprises:
either a step of reductive amination between an aldehyde reagent or an amine reagent, one of the reagents possibly bearing a precursor of the 8-hydroxyquinoline motif and the other possibly bearing a precursor of the polyamine chain,
or a step of nucleophilic substitution of an amine reagent bearing a precursor of the polyamine chain on an electrophilic agent bearing a precursor of the 8-hydroxyquinoline motif,
or a step of Michael addition between an alkene reagent bearing a precursor of the 8-hydroxyquinoline motif and an amine reagent bearing a precursor of the polyamine chain.

In particular, said precursors of the 8-hydroxyquinoline motif comprise, in the alpha position of the nitrogen atom constituting the 8-hydroxyquinoline cycle, a functional group suited to reacting with the second reagent bearing the precursor of the polyamine chain.

For the preparation of a Quilamine of formula (I) in which a is equal to 0 and b is equal to 0 (denoted as Quilamine HQ′0), the preparation method comprises the following steps:
(i) reduction amination between a primary amine which is a carrier of the 8-hydroxyquinoline motif of formula (II)

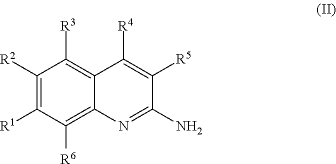

in which R¹, R², R³, R⁴, R⁵ and R⁶ are as defined in the formula (I) and of which the thiol, hydroxyl, primary and secondary amine functions, which can be present, are if necessary protected by appropriate protective groupings and a carboxaldehyde carrier of a polyamine chain of formula (II′)

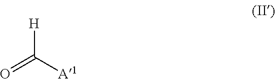

in which A′¹ represents an amine hydrocarbon group corresponding to A¹ as defined here above, and the primary and secondary amine functions of which are protected by appropriate protective groupings;
(ii) then deprotection of the protected functions in one or more steps.

At the step (i), the reductive agent is chosen from among hydrogen (H₂) in the presence of a catalyst (palladium on activated carbon for example), a hydride (NaBH₄, NaBH₃CN or sodium triacetoxyborohydride NaBH(OAc)₃ for example), and hydrogen donor (formic acid or one of its salts for example). Preferably, the invention uses sodium triacetoxyborohydride.

The thiol, hydroxyl primary and secondary amine functions can be protected by means of a well-known protective grouping that enables them to be made inactive relative to the reductive amination reaction. The deprotection reactions corresponding to the different protected functions are also known per se by those skilled in the art.

By way of examples of protective groupings of hydroxyl functions, we may cite the acetyl group (Ac), the silyl or methyl derivatives. These functions can be deprotected respectively by an acid treatment, a fluoride anion or boron tribromide. By way of examples of protective groupings of the primary and secondary amine functions of these polyamine chains, we may cite for example tertiobutyloxycarbonyl (Boc), benzyloxycarbonyl or the benzyl grouping. These functions can be deprotected respectively by an acid treatment with trifluoroacetic or hydrochloric acid, by hydrogenolysis in the presence of dihydrogen and palladium. When the 8-hydroxyquinoline motif comprises primary or secondly amine functions it is possible to protect and deprotect in the same way.

Carboxaldehyde which is a carrier of a polyamine chain, having the formula (II′) can be obtained from a hydrocarbon monomer constituting the polyamine chain (for example B¹, B², B³ in the case of the formula (X)) comprising a terminal hydroxyl primary function and a terminal amine primary function, i.e. at the end of the chain. The polyamine chain can be obtained by an N-substitution on the primary amine of the first monomer, of a second constituent monomer of the polyamine chain and bearing a nitrile function; reduction of the nitrile function into primary amine; protection of the appropriate functions (secondary amine in particular); then repetition if necessary of these steps until the desired polyamine chain is obtained. Carboxaldehyde is finally obtained by oxidizing the primary hydroxyl function of the intermediate hydroxypolyamine obtained.

The following diagram illustrates a method of this kind for preparing a HQ′0 (HQ0-44, 8″) type Quilamine:

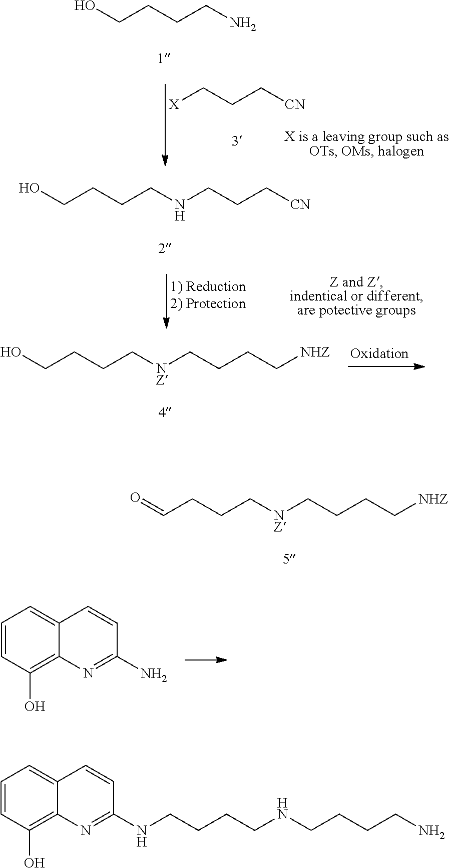

For the preparation of a Quilamine of formula (I) in which a is equal to 0, b is equal to 1 and L represents a CH₂ group (denoted as HQ′1), the method of preparation comprises the following steps:
(i) reductive amination between a carboxaldehyde reagent bearing the 8-hydroxyquinoline motif, of formula (III)

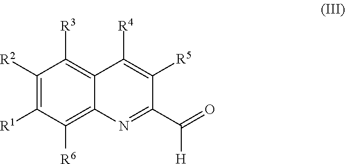

in which R¹, R², R³, R⁴, R⁵ and R⁶ are as defined in the formula (I), and the thiol, hydroxyl primary and secondary amine functions, which can be present, are if necessary protected by appropriate protective groupings;
and a primary amine bearing a polyamine chain of formula (III′)

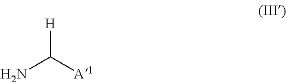

in which A′¹ is as defined in the formula (II′);
(ii) then deprotection of the protected functions in one or more steps.

The reductive agent can be chosen from among those used in the method for preparing as described here above. Preferably, the invention uses sodium triacetoxyborohydride.

The thiol, hydroxyl, primary and secondary amine functions can be protected and deprotected as indicated in the above method of preparation.

The primary amine bearing a polyamine chain, of formula (III′), can be obtained by a method for preparing similar to that of the carboxaldehyde of formula (II′) described further above, using a hydrocarbon monomer constituting the polyamine chain (for example B¹, B², B³ in the case of the formula (X)) comprising two primary amine functions. One of the amine functions is protected preliminarily by a protective grouping. Then, the polyamine chain can be obtained by an N-substitution, on the unprotected primary amine of the first monomer, of a second monomer that constitutes the polyamine chain and bears a nitrile function, reduction of the nitrile function into a primary amine; then protection of the appropriate function (secondary amine especially) before a possible repetition of these steps until the desired polyamine chain is obtained.

The scheme below illustrates a method for preparing HQ′1 (HQ1-44, 8) type Quilamine according to this embodiment using commercial hydroxyquinoline carbaldehyde 6:

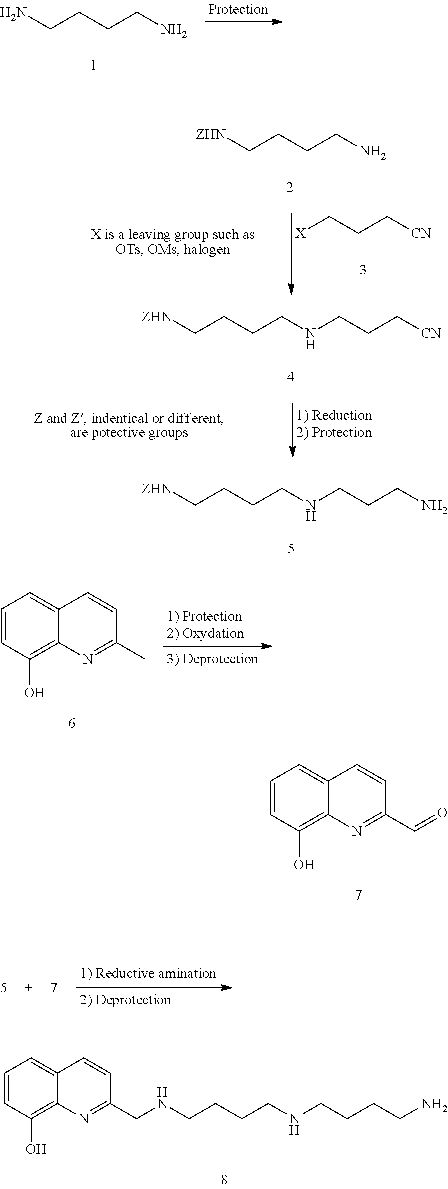

For the preparation of a Quilamine of formula (I) in which a is equal to 0, b is equal to 1 and L represents a CH₂—CH₂ group (denoted as HQ′2) three methods for preparing can be envisaged.

According to a first variant, the method for preparing Quilamine HQ′2 comprises the following steps:
(i) reductive amination between an aldehyde bearing the 8-hydroxyquinoline motif, of formula (IV)

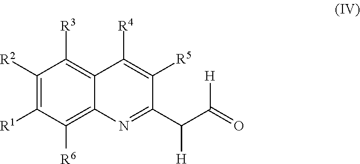

in which R¹, R², R³, R⁴, R⁵ and R⁶ are as in the formula (I), and of which the thiol, hydroxyl, primary and secondary amine functions, which may be present, are protected if necessary protected by appropriate protective groupings;
   a primary amine bearing a polyamine chain, of formula (III′) as defined here above;
(ii) then deprotection of the protected functions in one or more steps.

The reducing agent can be chosen from among those used in the method for preparing described here above. Preferably sodium triacetoxyborohydride is used.

The thiol, hydroxyl, primary and secondary amine functions can be protected and deprotected as indicated in the above method for preparing.

According to a second variant, the method for preparing Quilamine HQ′2 comprises the following steps:
(i) Michael addition between an alkene carrier of the 8-hydroxyquinoline motif of formula (V)

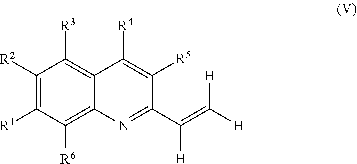

in which R¹, R², R³, R⁴, R⁵ and R⁶ are as defined in the formula (I) and of which the thiol, hydroxyl, amine functions, which can be present, are possibly protected by appropriate protective groupings;
   and a primary amine carrier of a polyamine chain, of formula (III′) as defined here above;
(ii) then deprotection of the protected functions in one or more steps.

The reducing agent can be chosen from among those used in the method for preparing described here above. Preferably, sodium triacetoxyborohydride is used.

The thiol and primary and secondary amine functions can be protected and deprotected as indicated in the previous method for preparing.

According to a third variant, the method for preparing Quilamine HQ′2 comprises the following steps:
(i) nucleophilic substitution of an electrophilic reagent bearing the 8-hydroxyquinoline motif, of formula (VI)

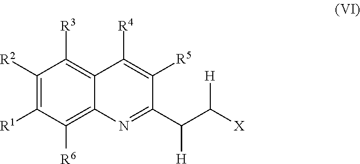

in which R¹, R², R³, R⁴, R⁵ and R⁶ are as defined in the formula (I) and of which the thiol, hydroxyl, primary and secondary amine functions, which can be present, are protected if necessary by appropriate protective groupings;
X represents a good leaving group, such as tosylate (OTs) or mesylate (OMs), or a halogen;
   by a primary amine that is a carrier of a polyamine chain, of formula (III′) as defined here above;
   (ii) then deprotection of the protective functions in one or more steps.

The following scheme illustrates a method for preparing a type HQ′2 Quilamine according to each of the three variants of this embodiment from commercial hydroxyquinaldine 6:

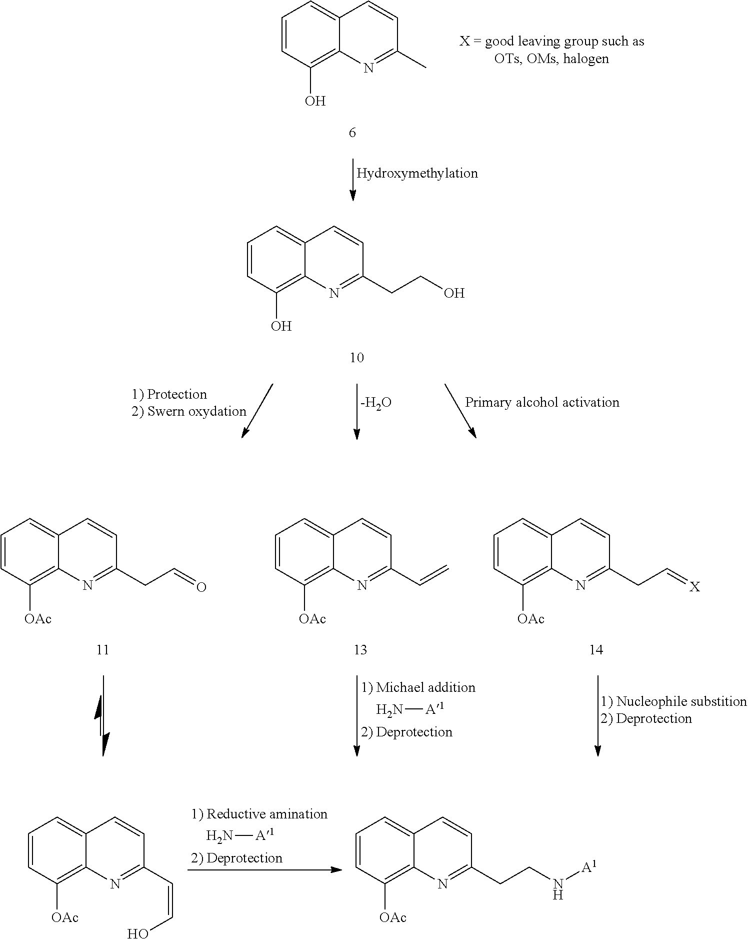

The hydroxymethylation of the compound 6 can be obtained by making it react with a butyllithium and then paraformaldehyde.

To prepare a Quilamine of formula (I) in which a is equal to 0, b is equal to 1 and L represents a group C═O (denoted as HQ′(CO)) this can be obtained by any classic reaction whatsoever for obtaining an amide from a primary amine. By way of an example, it is possible to cite the reactions of peptic coupling such as the reaction of a carboxylic acid bearing the 8-hydroxyquinoline motif, of formula (VII)

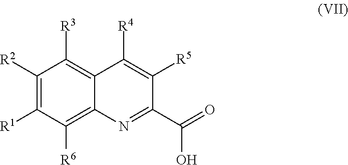

in which R¹, R², R³, R⁴, R⁵ and R⁶ are as defined in the formula (I) and of which the thiol, hydroxyl, primary and secondary amine functions, which can be present, are protected if necessary by appropriate protective groupings.
   with a primary amine that is a carrier of a polyamine chain, of formula (III′) as defined here above, in the presence of a coupling agent after protection of the other amines which can be present in the polyamine chain.

Among the coupling agents that can be used, we can cite 1-ethyl-3-(3-dimethylaminopropyl) carbodiimide (EDC or EDCI) and N,N′-dicyclohexylcarbodiimide (DCC). It is possible to add N-Hydroxybenzotriazole (HOBt) to the rectional medium in order to activate the reaction.

The scheme below illustrates the method for preparing a Quilamine HQ′(CO) according to this embodiment:

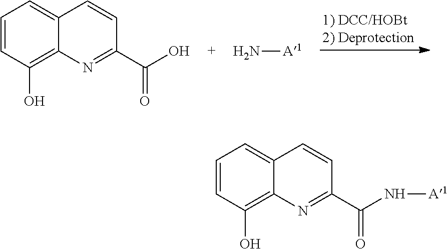

For the preparation of a Quilamine of formula (I) in which a is equal to 0, b is equal to 1 and L represents a group C═S (denoted as HQ′(CS)), this can be obtained by (i) thionation, by means of the Lawesson reagent or diphosphorus pentasulfide (P₄S₁₀), a Quilamine HQ′(CO), of which the thiol, hydroxyl, primary and secondary amine functions that can be presented are protected if necessary by appropriate protective groupings, and then (ii) deprotection of the protection functions. The thiol, hydroxyl, primary and secondary amine functions are preferably protected and deprotected as indicated in the above method of preparation.

The diagram below illustrates a method for preparing a Quilamine HQ′(CS) according to this embodiment:

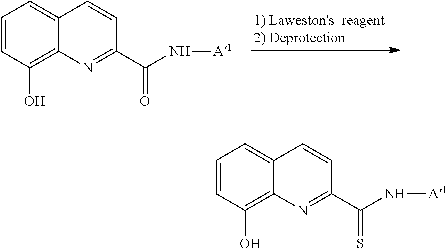

In a sixth embodiment, the method of the invention relates to the preparation of a Quilamine of formula (I) in which a is equal to 1, c and c′ are identical and L and L′ are identical, D¹ and D² are identical, and/or R¹, R², R³, R⁴, R⁵ and R⁶ are identical to R′¹, R′², R′³, R′⁴, R′⁵ and R′⁶ respectively.

Said Quilamine according to the invention can be obtained by (i) reductive amination of two equivalents of a carbaldehyde bearing an 8-hydroxyquinoline motif with an equivalent of a primary diamine bearing a polyaminate chain, the primary and secondary amine functions of which have been protected, then (ii) deprotection of the protection functions, said carbaldehyde being in position 2 of the 8-hydroxyquinoleine motif. The primary and secondary amine functions of the polyamine chain can be protected and deprotected as taught here above.

By way of usable carbaldehydes, we can cite the compounds corresponding to the following formula (VIII), their salts, solvates or conjugate forms:

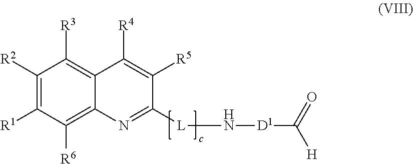

in which: L, c, D^{1,} R¹, R², R³, R⁴, R⁵ and R⁶ are as defined in the formula (I).

The reagent levels used in each of the above embodiments can be chosen and optimized to obtain the best yields.

The following examples serve to illustrate the different aspects of the invention.

### EXAMPLES

### Example 1

### Preparation of Quilamine 8′ (2-[4-(4-aminobutyl)aminobutyl]aminomethylquinolin-8-ol)

### 1) Preparation of the reagent 5′ (N⁴,N⁸-di-tert-butyloxycarbonylspermidine)

### Synthesis of tertiobutyl (4-aminobutyl)carbamoate 2′

4.4 g of 1.4 diaminobutane (0.05 mol) are dissolved in 110 ml of a solution of triethylamine and methanol (10% by volume of TEA in methanol) under argon and at 0° C. A solution of di-terbutyl dicarbonate (3.63 g, 0.017 mol) and methanol (10 ml) is added dropwise into the mixture under heavy stirring. The mixture is stirred at ambient temperature over one night. The solvents are removed under vacuum to obtain an oily residue which is dissolved in dichloromethane (100 ml) and washed two times with 100 ml of an aqueous solution of sodium hydroxide (10% by weight of NaOH in water).

The organic phase is dried and rid of solvent under vacuum and the product is purified by chromatography (1:10:89 NH₄OH:MeOH:CHCl₃). The product 2′ obtained is a translucent oil (yield: 81%). R_{f} 0,4 (NH₄OH/MeOH/CHCl₃).

### Here Here

NMR¹H (300 MHz, CDCL₃) δ 4.71 (s, 1H, NHCO), 3.10 (dt, J₁=6 Hz, 2H, CH₂); 2.69 (t, J₁=6 Hz, 2H, CH₂); 1.57-1.44 (m, 4H, 2CH₂); 1.42 (s, 9H, CH₃).

NMR¹³C (75 MHz, CDCl₃) δ 156.2; 79.2; 41.9; 40.8; 30.8, 28.5; 27.6.

### Synthesis of tertiobutyl [4-(3-cyanopropylamino)-butyl]-carbamate 4′

To a solution of amine protected by tertio-butoxycarbonyl 2′ (2.1 g, 0.01 mol) and anhydrous acetronitril (75 ml), 5.14 g potassium carbonate is added. The suspension is mixed at ambient temperature for 10 minutes. 4-bromobutyronitrile 3′ (1.65 g, 0.01 mol) is added to the mixture and the entire solution is stirred at 50° C. for 24 hours. The mixture is filtered to remove a major part of the inorganic salts and the acetonitrile is removed under vacuum. The oily residue obtained is purified by flash chromatography (1:5:94 NH₄OH:MeOH:CHCl₃). The product 4′ obtained is a translucent oil (yield: 76%). R_{f}: 0.5 (1:10:89 NH₄OH:MeOH:CHCl₃).

NMR¹H (300 MHz, CDCl₃) δ 4.77 (s, 1H, NHCO); 3.11 (q, J₁=6 Hz, 2H, CH₂); 2.73 (t, J=6.9 Hz; 2H, CH₂); 2.61 (t, J=6.6 Hz, 2H, CH₂); 2.44 (t, J=7.2 Hz, 2H, CH₂); 1.80 (quintuplet, J=6.9 Hz, 2H, CH₂); 1,55-1.46 (m, 4H, 2CH₂); 1.43 (s, 9H, 3CH₃).

NMR¹³C (75 MHz, CDCl₃) δ 156.4; 119.8; 78.9; 76.4; 49.3; 48.0; 40.4; 28.42 (3C); 27.8; 27.4; 25.8; 14.9.

### Synthesis of tertiobutyl (4-aminobutyl-(4-tertiobutoxycarbonylaminobutyl)carbamate 5

### a) Protection of Amine 4′

Aminonitrile 4′ (1.74 g, 6.8 mmol) is dissolved in 40 ml of a solution of methanol and triethylamine (10% by volume of TEA in methanol). The di-tertio-butyldicarbonate (3.63 g, 0.017 mol) is added dropwise to the mixture under stirring.

The mixture is stirred at ambient temperature for one night. The solvents (MeOH and TEA) are evaporated under vacuum to obtain an oily residue which is dissolved in dichloromethane (100 ml) and washed twice with 30 ml of an aqueous solution of hydrogenocarbonate (saturated solution in water) and twice with 30 ml of water.

The organic phase is dried with Na₂SO₄, filtered and purified by flash chromatography (80:20, dichloromethane: ethyl acetate). The intermediate product obtained is a translucent oil (95%).

NMR¹H: (300 MHz, CDCl₃) δ 4.57 (s, 0.8H, NHCO); 3.29 (t, J=6.9 Hz, 2H, CH₂); 3.03-3.23 (m, 4H, 2CH₂); 2.34 (t, J=7.2 Hz, 2H, CH₂); 1.87 (t, J=6.9 Hz, 2H, CH₂); 1.46-1.61 (m, 4H, 2xCH₂); 1.45 (s, 9H, CH₃); 1.43 (s, 9H, CH₃).

NMR¹³C (75 MHz, CDCl₃) δ 156.1; 157.7, 119.4; 80.1; 79.3, 47.2, 45.8, 40.2, 28.5, 27.8, 27.5, 25.6, 24.6, 14.8.

### b) Reduction of the Nitrile Function and Obtaining of the Product 5′

Raney nickel (50% by weight in an aqueous solution) is washed in ethanol (and kept permanently in a wet state in solvent because the compound is pyrophoric). The intermediate product obtained at the previous step (2.00 g, 5.6 mmol) and NH₄OH are added. Argon is made to bubble for 20 minutes. The suspension is hydrogenated at 20 bars for 24 hours, Raney nickel is filtered on Celite® (Raney nickel is kept permanently in a wet state by means of ethanol). The ethanol and NH₄OH are evaporated under vacuum and the oily residue is dissolved in CH₂Cl₂ and washed with an aqueous solution of 10% in weight of NaOH (3*50 ml). The organic phase is dried by means of Na₂SO₄, filtered and the solvent is evaporated under vacuum to obtain the product 5′ (98%).

NMR¹H: (300 MHz, MeOD) δ 3.21 (t, 4H, J=7.2 Hz,); 3.04 (t, 2H, J=6.9 Hz, 2CH₂); 2.66 (t, 2H, J=7.05 Hz,); 1.62-1.51 (m, 8H); 1.46 (s, 9H), 1.43 (s, 9H).

NMR¹³C (75 MHz, MeOD) δ 158.5, 157.4, 80.8, 79.8, 42.2, 40.9, 30.8, 28.7, 28.3, 27.1, 26.6.

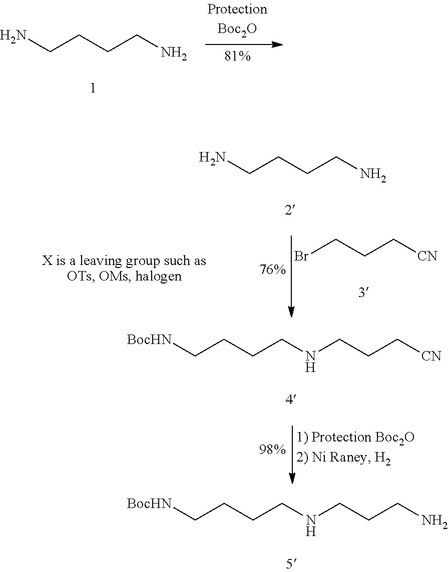 [SMILES: NCCCCN ; NCCCCN ; N#CCCCBr ; "X is a leaving group such asOTs, OMs, halogen" ; CC(C)(C)OC(=O)NCCCCNCCCC#N ; CC(C)(C)OC(=O)NCCCCNCCCN]

### 2) Preparation of reagent 7 (8-hydroxyquinoline-2-carbaldehyde)

The reagent 7 is a commercially available reagent. It can be obtained from the product 6 according to the following rectional scheme:

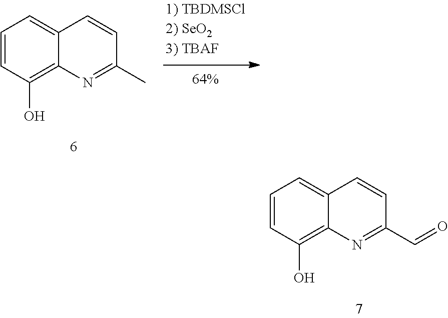

### 3) Preparation of Quilamine 8′ (2-[4-(4-aminobutyl)aminobutyl]aminomethylquinolin-8-ol)

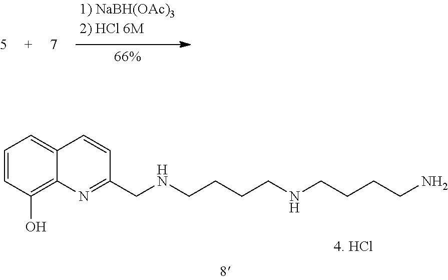 [SMILES: NCCCCNCCCCNCc1ccc2cccc(O)c2n1]

An equivalent of 8-hydroxyquinoline-2-carbaldehyde 7 is added to a solution of polyamines 5′ (1.2 eq) in 1.2-dichloroethane (10 ml) at ambient temperature with stirring under argon.

After 15 minutes, three equivalents of sodium triacetoxyborohydride are added and the mixture is stirred for 12 hours until the aldehyde is consumed (followed by thin-layer chromatography). The reaction is stopped by adding 10 ml of an aqueous solution of NaOH (1 mol/liter). The mixture is stirred for 20 minutes more and the product is extracted with dichloromethane (3 times), dried on sodium sulfate and concentrated under reduced pressure. The residue is purified by chromatography on silica gel (chloroform/methanol/ammonia: (94:5:1)). A pale yellow oil is obtained (91%).

IR (KBr): ν=3053, 2982, 2934, 1706, 1683, 1575, 1507, 1475, 1455, 1420, 1391, 1366, 1265, 1168, 1047 cm⁻¹.

¹H NMR (400 MHz, [D₄]MeOD): δ=1.35-1.69 (m, 26H, 8CH₂ 6CH₃), 2.77 (bs, 2H, CH₂), 3.03 (t, 2H, CH₂, J=6.8 Hz), 3.13-3.26 (m, 4H, 2CH₂), 4.14 (s, 2H, CH₂), 7.10 (dd, H, J=7.5 Hz, J=1.3 Hz), 7.34 (dd, H, J=8.3 Hz, J=1.3 Hz), 7.41 (dd, H, J=8.1 Hz, J=7.6 Hz), 7.45 (d, H, J=8.5 Hz), 8.20 (d, H, J=8.5 Hz).

¹³C NMR (100 MHz, [D₄]MeOD): δ=26.5, 27.0, 27.2, 28.4, 28.7 (3C), 28.8 (3C), 40.9, 48.1, 49.9, 54.9, 79.8, 80.8, 112.2, 118.8, 122.1, 128.3, 129.4, 137.9, 139.2, 154.2, 157.4 (2C), 158.5.

High-resolution mass spectrometry (MALDI): calculated C₂₈H₄₄N₄O₅ [M+H]⁺517.3384. found 517.3386 HERE HERE

### Deprotection:

The diprotected compound is put into solution in 5 ml of ethanol and then 6 ml of an aqueous solution of hydrochloric acid (6 mol/L) is added at 0° C. The mixture is shaken for 24 hours and the solvent is evaporated. The solid is taken up in a minimum of ethanol and the precipitant is filtered under vacuum and pump dried for six hours (73%).

Melting point above 250° C.

IR (KBr): ν=3384, 2957, 2798, 1642, 1606, 1551, 1513, 1461, 1420, 1398, 1344, 1302, 1252-1055, 841 cm⁻¹.

¹H NMR (400 MHz, D₂O): δ=1.76-2.04 (m, 8H, 4CH₂), 3.06-3.22 (m, 6H, 3CH₂), 3.38 (dd, 2H, J=7.75 Hz, J=7.45 Hz, CH₂), 4.78 (s, 2H, CH₂), 7.36 (dd, 1H, J=7.45 Hz, J=1.49 Hz, H₇ₐᵣ), 7.60 (dd, 1H, J=8.30 Hz, J=1.49 Hz, H₅ₐᵣ), 7.65 (dd, 1H, J=8.30 Hz, J=7.45 Hz, H₆ₐᵣ), 7.76 (d, 1H, J=8.60 Hz, H₃ₐᵣ), 8.64 (d, 1H, J=8.60 Hz, H₄ₐᵣ) ppm.

¹³C NMR (100 MHz, D₂O): δ=22.7, 22.8 (2C), 24.0, 38.9, 46.9, 47.0, 47.3, 49.7, 114.1, 119.4, 121.1, 128.9, 129.3, 134.5, 141.8, 148.5, 149.5 ppm.

High-resolution mass spectrometry (MALDI): C₁₈H₂₉N₄O [M+H−4HCl]⁺: Calculated: 317.2336. Found: 317.2348.

Elementary analysis: C₁₈H₃₂Cl₄N₄O 1.2H₂O: Calculated: C, 44.68; H, 7.17; N, 11.58. Found: C, 44.52; H, 6.97; N, 11.57.

### Example 2

### Preparation of an HQ′(CO) Quilamine

### Synthesis of N-(4-((4-aminobutyl)amino)butyl)-8-hydroxyquinoline-2-carboxamide

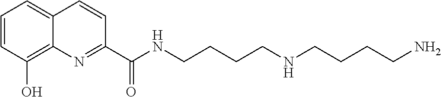 [SMILES: NCCCCNCCCCNC(=O)c1ccc2cccc(O)c2n1]

104 mg of 2-acid 8-hydroxyquinoline (0.552 mmol) are dissolved in anhydrous dichloromethane under argon. 80.8 mg of hydroxybenzotriazole (HOBT) (0.607 mmol) and 122 mg of dichlohexyl carbodiimide (DCC) (0.607 mmol) are stirred at 0° C. The solution is stirred for one hour and the amine 4′ is added (200 mg, 0.552 mmol). The mixture is stirred for 24 hours. The solvents are evaporated under vacuum and the residue is dissolved in dichloromethane, washed with NaHCO₃, dried on Na₂SO₄ and filtered. The solvent is removed under vacuum and the product is purified by flash chromatography (dichloromethane: methanol, 97:3). The protected product obtained is a brown solid (yield: 99%).

NMR¹H (400 MHz, MeOD) δ 8.39 (d, J=8.5 Hz, 1H); 8.18 (d, J=8.5 Hz, 1H); 7.53 (dd, Jₜ=8.2 Hz, J_{d}=7.6 Hz, 1H); 7.42 (dd, J=8.2 Hz, J=1.0 Hz, 1H); 7.16 (dd, J=7.6 Hz, J=1.0 Hz 1H); 3.51 (t, J=6.3 Hz, 2H); 3.19 (t, J=7.2 Hz, 2H); 3.01 (t, 2H, J=6.7 Hz,); 1.74-1.31 (m, 26H).

NMR ¹³C (100 MHz, MeOD) δ 166.6, 158.5, 157.4, 155.0, 148.8, 138.8, 138.4, 131.4, 130.5, 119.9, 118.9, 112.7, 80.8, 79.8, 48.1, 40.9, 40.3, 28.8, 28.7, 28.3, 27.9, 27.0.

### Deprotection:

The protected product (100 mg, 0.188 mmol) is dissolved in 1 ml of ethanol and then an excess of an aqueous solution of hydrochloric acid (6 mol/L) is added. The solution is mixed at ambient temperature for 24 hours, the solvents are evaporated under vacuum. The product obtained is a yellow solid (60 mg, yield 96%).

NMR¹H (400 MHz, D₂O) δ 8.16 (d, J=8.7 Hz, 1H); 7.74 (d, J=8.7 Hz, 1H); 7.45 (dd, J=8.4 Hz, J=7.7 Hz, 1H); 7.29 (dd, J=1.1 Hz, J=1.1 Hz, 1H); 7.07 (dd, J=1.1 Hz, J=7.7 Hz, 1H); 3.48 (t, J=6.6 Hz, 2H); 3.23-3.03 (m, 6H); 1.91-1.73 (m, 8H).

NMR¹³C (100 MHz, MeOD) δ 165.5, 150.9, 145.8, 138.4, 135.4, 129.5, 129.4, 118.9, 118.3, 112.2, 47.9, 46.8, 38.9, 38.8, 25.7, 23.9, 23.2, 22.7.

The physical/chemical and biological properties of Quilamines according to the present invention have been studied and the results of these studies are presented here below with reference to the figures in which:

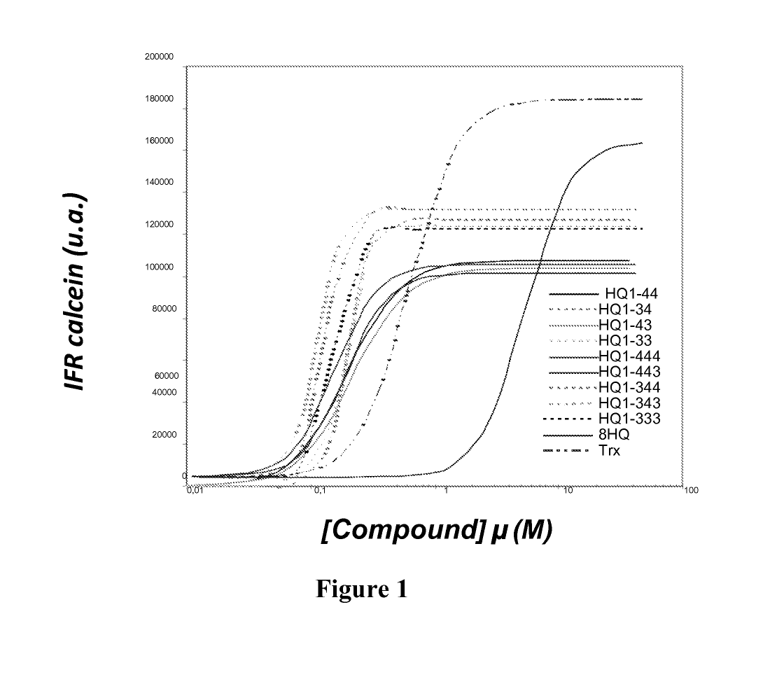

FIG. 1 presents the contribution of the polyamine chain in the chelation of iron by the Quilamines according to the invention;

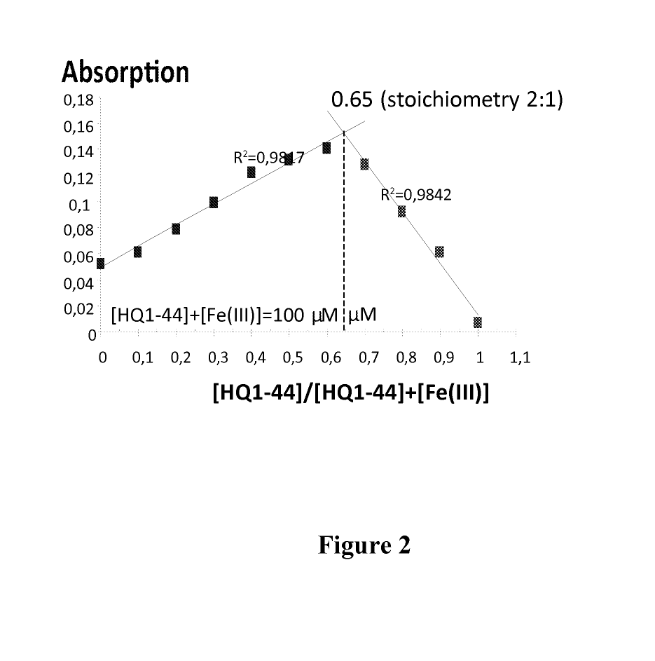

FIG. 2 presents the stoichiometry of the interaction of Quilamine HQ1-44 with iron (L₂:Fe₁);

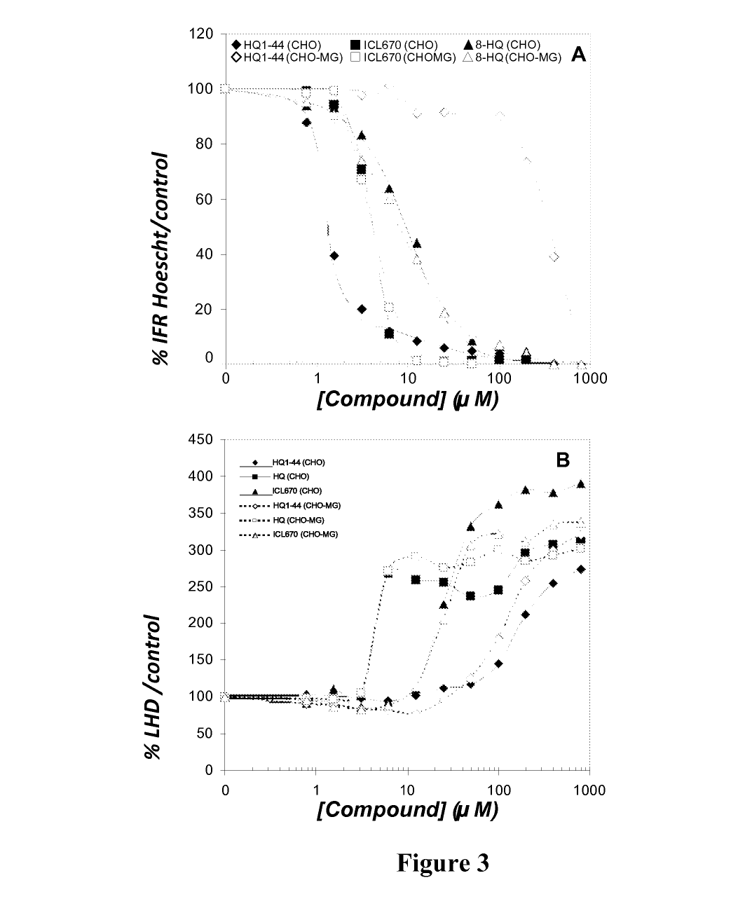

FIG. 3 presents the effects of Quilamine HQ1-44 (⋄) and reference chelators, 8-hydroxyquinoline (HQ, □) and Deferasirox™ (ICL670, Δ) on the viability of hamster ovarian cells, the wild cell line (CHO, solid-line curves) and the deficient mutant cell line for the polyamine transport system (CHO-MG, dotted curve);

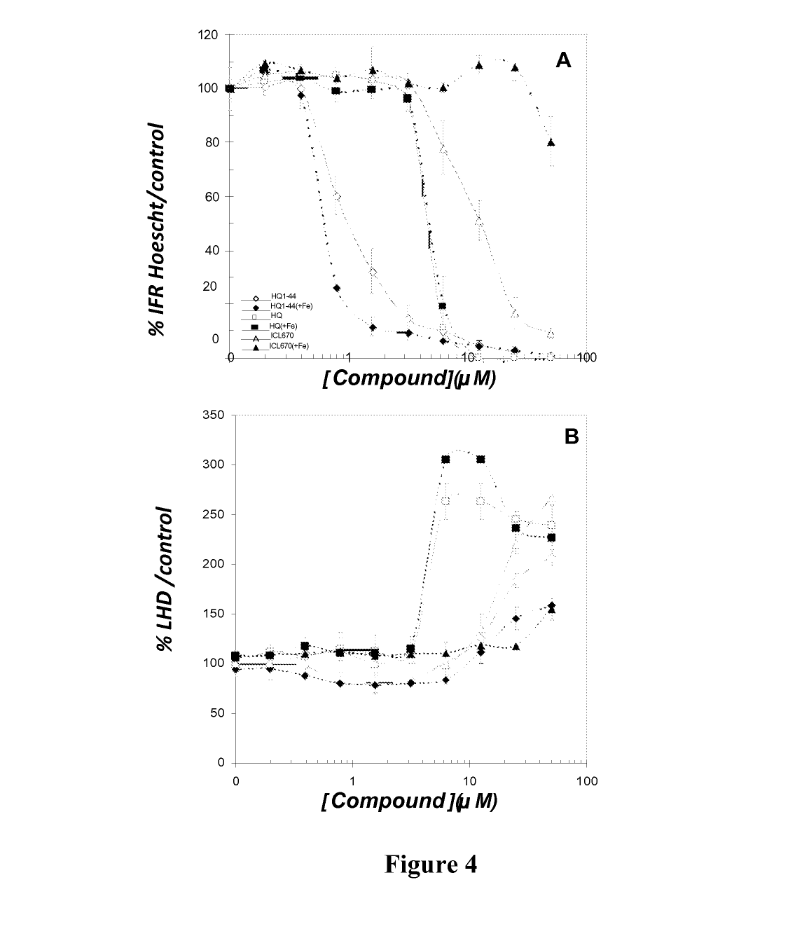

FIG. 4 presents the influence of 20 micromolar exogenous iron (dotted curves) on the viability of hamster ovarian cells (CHO), exposed to different concentrations in compounds, namely Quilamine HQ1-44 (⋄) and reference chelators, 8-hydroxyquinoleine (HQ, □) and Deferasirox™ (ICL670, Δ);

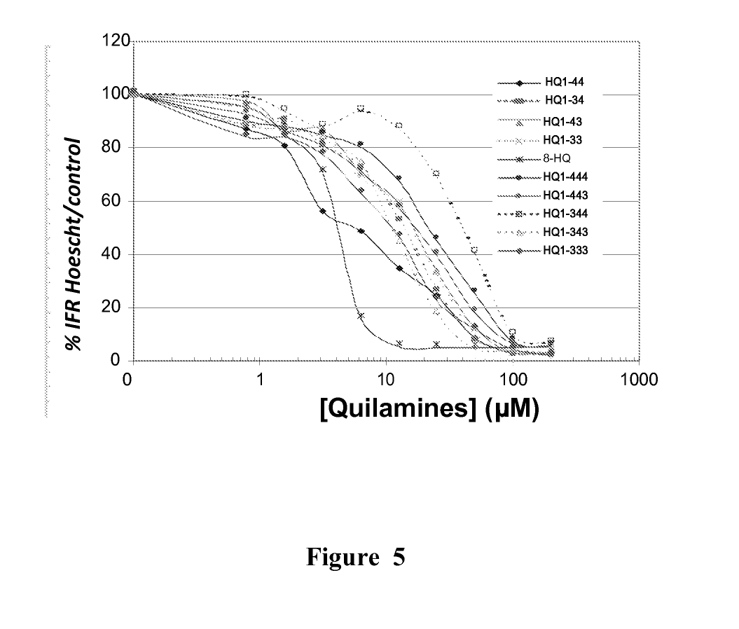

FIG. 5 presents the effects of the Quilamines and the reference chelators, the 8-hydroxyquinoleine (8-HQ) on the viability of tumor cells derived from human hepatocarcinoma (HepG2);

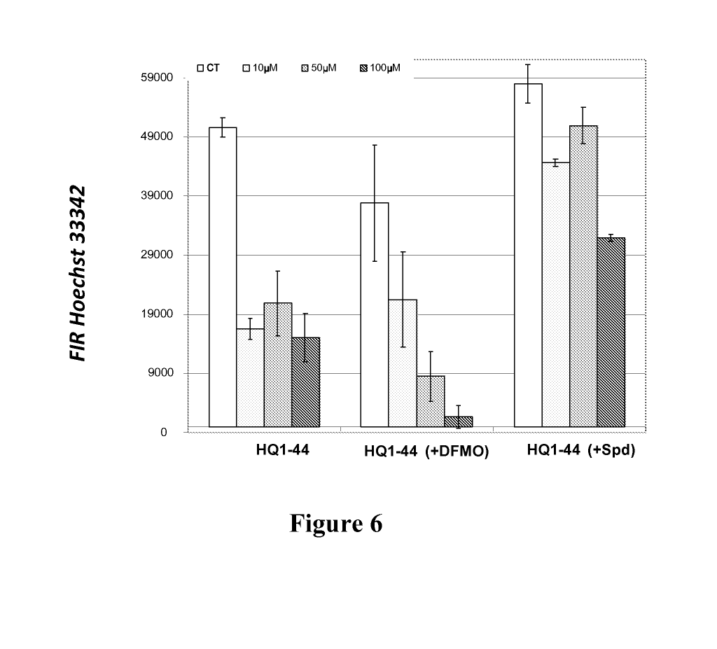

FIG. 6 presents the influence of PTS on the viability of human hepatocarcinoma cells (HepG2) treated with Quilamine HQ1-44;

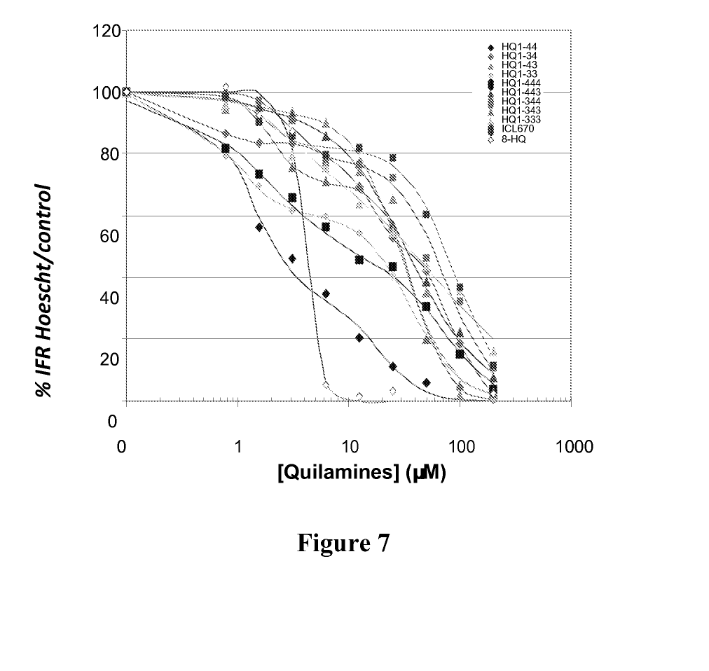

FIG. 7 presents the effects of Quilamines and reference chelators, 8-hydroxyquinoleine (8-HQ) and Deferasirox™ (ICL670) on the viability of tumor cells derived from adenocarcinomas of the human colon (Caco-2);

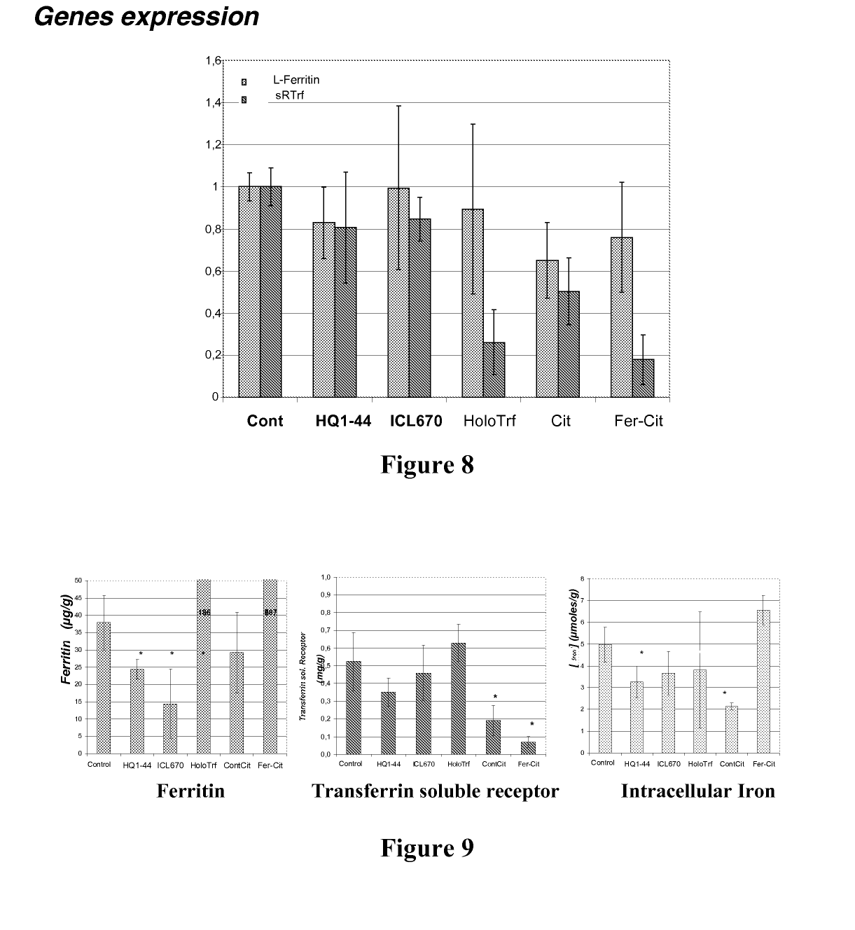

FIG. 8 presents the effect of Quilamine HQ1-44 and reference chelators, Deferasirox™ (ICL670) on the expression of genes of iron metabolism in adenocarcinoma cells of the human colon (Caco-2);

FIG. 9 presents the effect of Quilamine HQ1-44 and of the reference chelator, Deferasirox™ (ICL670) on the iron metabolism of adenocarcinoma cells of the human colon (Caco-2);

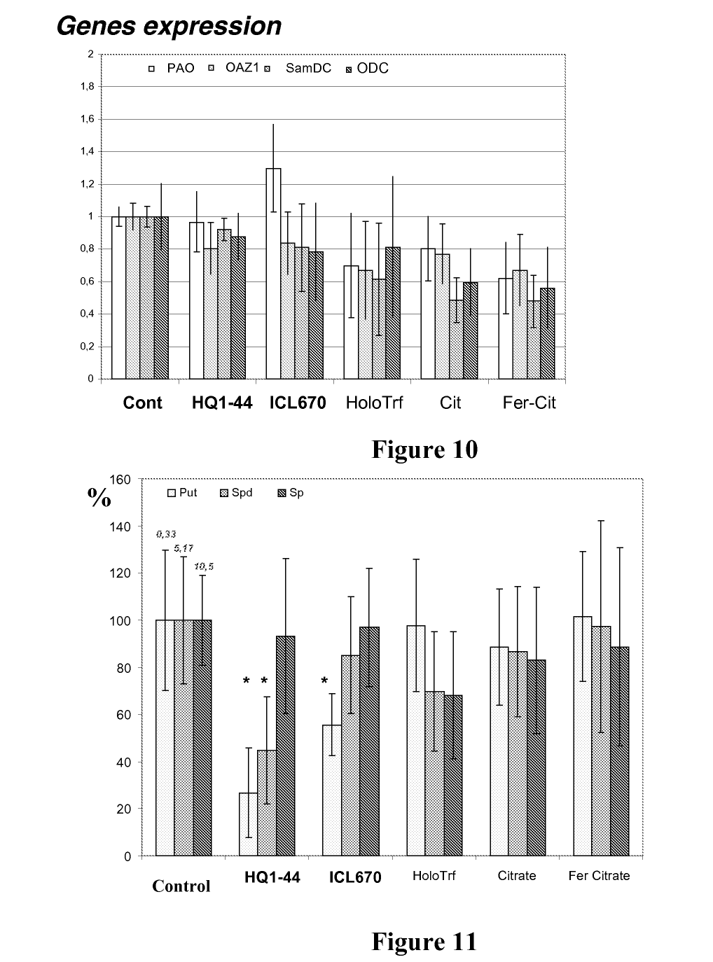

FIG. 10 presents the effect of Quilamine HQ1-44 and of the reference chelator, Deferasirox™ (ICL670) on the expression of genes of the polyamine metabolism of adenocarcinoma cells of the human colon (Caco-2);

FIG. 11 presents the effect of Quilamine HQ1-44 and of the reference chelator, Deferasirox™ (ICL670) on the polyamine metabolism of adenocarcinoma cells of the human colon (Caco-2).

### I-Physical-Chemical Properties of Quilamines

1. Capacity of Interaction with Labile Iron: Calcein Test (FIG. 1)

The Quilamines HQ1-34, HQ1-344, HQ1-343, HQ1-33, HQ1-333, HQ1-44, HQ1-43, HQ1-444, HQ1-443 according to the invention were synthesized and tested. For each Quilamine, its capacity to shift iron (III) complexated with calcein was tested. Calcein is a fluorescent molecule which has a pFe3+ value of 20.3 with pH 7.4 close to the value measured for ligands of low molecular weight present in human or animal cells and complexating the labile iron pool, such as citrate which has a pFe³⁺ value of 19.3 with pH of 7.4 (LIP). The fluorescence of calcein is detected on a microplate, in a concentration of 0.1 micromole per liter, in solution in a HEPES buffer (4-(2-hydroxyethyl)-1-piperazine ethane sulfonic acid) with pH 7.4, in a Fusion (Packard) type a microplate fluorescence reader, under excitation at 450 nanometers and analysis of the fluorescence emission at 515 nanometers. The fluorescence of calcein is extinguished in the presence of iron(III) in the form of hydrochloride (chloride) with a concentration of 1 micromole per liter. The addition of Quilamine in different concentrations ranging from 0.01 to 40 micromoles per liter prompts the shifting of iron(III) complexated with calcein towards Quilamine, resulting in a restoration of the fluorescence of the calcein. The dose-effect curves expressing the percentage of restoration of fluorescence of calcein (relative to the fluorescence of the free molecule in the absence of iron), for each Quilamine concentration, makes it possible to deduce the Quilamine concentration prompting the 50% shift of Fe(III) from calcein and the restoration of its associated fluorescence (CE50). These concentration values CE50 are inversely proportional to the chelating capacity of the chelators and the aptitude to mobilize iron from the LIP of the organism.

Comparatively, measurements were made, under the same conditions, of the efficacy in shifting iron(III) complexated with calcein of the two prior-art chelators, 8-hydroxyquinoline and O-trensox, the latter being currently considered to be the chelator having the greatest affinity for iron.

Referring to FIG. 1, the tests showed that the Quilamines according to the invention have higher chelating capacity than the two chelators tested for chelating concentrations lower than 0.2 micromoles per liter. The comparison of the chelating efficiency of Quilamines relative to that of the basic chelating motif, 8-hydroxyquinoline, shows that the polyamine chain reinforces or boosts the capacity of interaction of this chelator with Fe(III). Furthermore, it was observed that Quilamines of the HQ1-3 (HQ1-34, HQ1-344, HQ1-343, HQ1-33, HQ1-333) type have a better chelating capacity as compared with Quilamines of the HQ1-4 (HQ1-44, HQ1-43, HQ1-444, HQ1-443) type.

2. Study of Interaction of Quilamine HQ1-44 with Iron (FIG. 2)

The interaction of Quilamine HQ1-44 with iron (Fe3+) was assessed by means of a characteristic transition at 580 nm, observed in the absorption spectrum of the complex of Quilamine HQ1-44 with Fe(III). The continuous variation method consist in measuring the quantity of complex formed between Quilamine and Fe(III) deduced from the value of absorption at 580 nm, in bringing about a variation of the ratio of the concentrations in Quilamine/Quilamine+Fe(III) for a total concentration of Quilamine+Fe(III) that is constant and equal to 100 micromoles per liter (Tris/HCl buffer 100 millimoles per liter, pH 7.4).

The continuous variation method showed that Quilamine HQ1-44 forms a complex with Fe(III) with a ligand/iron stoichiometry of 2/1 at physiological pH. Potentiometry measurements were made in thermostated titration cells at 25±0.1° C., using a Metrohm 702 SM Titrino titrator connected to a Metrohm 6.0233.100. The ligand solution is prepared in a concentration of ˜1.5×10⁻³ moles per liter, the solution of Fe³⁺ is made from FeCl₃ and titrated by complexation with a standard solution of EDTA (ethylenediaminetetracetic acid). The sample for the measurement contained approximately 0.03 millimoles per liter of ligand in a volume of 30 ml where the ionic strength is maintained at 0.1 mole per liter in using KNO₃ as a support electrolyte. During the titrations, Fe³⁺ is added to 0.45 or 0.9 equivalents of ligand. Each titration consists of 150-200 equilibrium points with pH varying from 2.0 to 11.5, and is repeated at least twice. The thermodynamic constants are computed with the software HYPERQUAD and the speciation diagram of the species is done by the Hyss program.

TABLE 1
Constants of stability (log K) of the HQ1-
44 (=L) iron complex (25.0° C., I = 0.1 M in KNO₃)

| species | L |
|---|---|
| FeL | 25.92 |
| FeLH | 6.65 |
| FeLH₂ | 2.88 |
| FeL(OH) | 7.37 |
| FeL₂ | 7.8 |
| FeL₂H | 10.2 |
| FeL₂H₂ | 8.0 |
| FeL₂H₃ | 8.2 |

The comparison of the pFe3+ value of HQ1-44 (27 at pH7.4) with that of o-Trensox which forms a complex having very high affinity with Fe(III), having a stoechiometery of 1/1 (pFe3+=29.5 measured in the same pH conditions) confirms the high chelating capacity of Quilamine HQ1-44. It must also be compared with that of the basic chelator motif, 8-hydroxyquinoline (pFe3+=20.6 measured in the same pH conditions), which confirms the reinforcement of the chelating capacity of this chelator by the polyamine chain of Quilamine HQ1-44.

### II-Biological Properties Relative to Healthy Hamster Ovarian Cells

### 1. Implication of PTS in the Capture of Quilamines

The in vitro efficacy of different Quilamines was tested on a wild cell line (CHO) of hamster ovarian cells having PTS and a mutant cell line CHO-MG without PTS. The two cell types CHO and CHO-MG were treated, 24 hours after being seeded in 96-well microplates, by concentrations of different Quilamines ranging from 0.1 to 400 micromoles per liter. The cytostatic (anti-proliferative) effects and cytotoxic effects of Quilamine after 72 hours' treatment of the cells were assessed respectively by counting cell nuclei after DNA marking by the Hoechst 33342 fluorescent intercalant dye and by analysis of membrane damage detected by the dosing of the lactate dehydrogenase activity (LHD) in the supernatants.

The results obtained with Quilamine HQ1-44 are shown in FIG. 3, FIG. 3A pertaining to the results on the cell viability and FIG. 3B pertaining the results on cell toxicity.

The IC50 inhibitor concentration corresponding to the concentration in Quilamines enables the inhibition at 50% of the cell proliferation was deduced from the dose-effect curves of numeration of the nuclei of the living cells on the CHO and CHO-MG cell lines. These dose-effect curves are sometimes bi-phase curves as in the case of Quilamine HQ1-44 which has an anti-proliferative constituent (without concomitant release of LDH in supernatant) in the range of concentrations of 0.4 to 3 micromoles per liter and a second cytotoxic constituent accompanied by membrane toxicity expressed by the release of LDH for concentrations of over 2 micromoles per liter. The lower the value of IC50, the greater the efficiency of the anti-proliferative effect of the tested chelators.

Furthermore, the ratio of the IC50 inhibitor concentrations found for the CHO-MG and the CHO cell lines (Ratio IC50 (CHO-MG/CHO)) was computed for several Quilamines according to the invention. This ratio makes it possible to estimate the selectivity of recognition of the different chelators by the PTS: the higher the ratio, the more clearly is the chelator recognized by the PTS. When this ratio is close to 1, the tested chelator is not recognized or is little recognized by the PTS.

The results were listed in table 2 here below and were compared with those obtained under the same conditions for two metal ion chelators of the prior art, 8-hydroxyquinoline and ICL670 (Deferasirox™ marketed by Novartis).

TABLE 2

| | IC50 (micromole.per liter) | | Ratio IC50 |
|---|---|---|---|
| Chelators | CHO | CHO-MG | (CHO-MG/CHO) |
| HQ1-44 | 1.4 | 344.5 | 249 |
| HQ1-444 | 6.3 | 239.5 | 38 |
| HQ1-33 | 5.7 | 117.9 | 21 |
| HQ1-333 | 15.1 | 277.0 | 18 |
| HQ1-443 | 20.2 | 257.5 | 13 |
| 8-hydroxyquinoline | 5.0 | 10.5 | 2 |
| ICL670 | 9.2 | 8.0 | 1 |

Influence of the polyamine transport system on the selectivity of the anti-proliferative action of Quilamines compared with that of the reference chelators, 8-hydroxyquinoline (8- HQ) and deferasirox ™ (ICL670).

The compound HQ1-44 is characterized by an IC50 of 1.4 micromoles per liter on this CHP cell line and 344 micromoles per liter on the CHO-MG cell line. This compound has the most efficacious anti-proliferative effect and is the one best recognized by the PTS.

Furthermore, the poly(n-butylamine) chain (HQ1-44 and HQ1-444) seem to be the most selectively recognized by PTS relative to the poly(n-propylamine) (HQ1-33).

Besides, the Quilamines of the invention that have been tested all have excellent recognition by the PTS as compared with a known 8-hydroxyquinoline and ICL670.

### 2. Efficacy of Quilamines

A comparison was made of the cytostatic or anti-proliferative action and the cytotoxic action for 72 h of treatment by Quilamine HQ1-44 and 8-hydroxyquinoline and ICL670, on the CHO cell lines in the presence and without the presence of exogenous iron, in the form of Fe(III) citrate in a concentration of 20 micromoles per liter. The results obtained are shown in FIG. 4, FIG. 4A relating to the results on cell viability and FIG. 4B relating to the results on cell toxicity.

In the absence of exogenous iron, it was observed that 8-hydroxyquinoline gave rise to a reduction of the number of viable cells associated with the release of LDH (cytotoxic effect). In particular, the 8-hydroxyquinoline exerted a cytotoxic effect as soon as a chelator concentration greater than or equal to 5 micromoles per liter was reached. In the presence of the exogenous iron, no modification was observed in the cytotoxic action of 8-hydroxyquinoleine.

The dose-effect curves for ICL670 are biphase curves as are those of HQ1-44 Quilamine. In the absence of exogenous iron, a cytostatic effect without membrane deterioration is observed for chelator concentration values ranging from 3 to 10 micromoles per liter and a cytotoxic effect is observed for chelator concentration values of over 10 micromoles per liter resulting in the release of LDH. After addition of exogenous iron, an inhibition is observed of the cytostatic and cytotoxic effect induced by this chelator.

For Quilamine HQ1-44, in the absence of exogenous iron a cytostatic effect without membrane deterioration is observed for chelator concentration values below 3 micromoles per liter (ranging from 0.4 to 3 micromoles per liter) and a cytotoxic effect is observed for chelator concentration values of over 3 micromoles per liter resulting in the release of LDH. After addition of exogenous iron, an improvement is observed in the anti-proliferative effect of Quilamine (IC50=0.6 micromoles per liter instead of 1.4 as measured previously) and a partial inhibition is observed in the cytotoxicity of this compound in chelator concentration values of over 3 micromoles per liter.

Quilamine HQ1-44 is therefore less toxic than 8-hydroxyquinoline and preserves an anti-proliferative action even in the event of serum iron overload unlike in the case of ICL670. This has a certain interest for treating proliferative diseases and/or diseases linked to iron overload in cells. The absence of inhibition of the anti-proliferative effect of Quilamine in the presence of exogenous iron suggests that the anti-proliferative action of Quilamine is not associated only with its capacity for depleting iron in cells. It could be also associated with the capacity of HQ1-44 for inhibiting polyamine metabolism such as the that shown here below in Caco-2 cells (§4).

### III-Biological Properties as Compared with Tumor Cells

### 1. Efficacy of Quilamines in Hepatocyte Tumor Cells of the HepG2 Cell Line

The efficacy of Quilamines was analyzed on hepatocyte tumor cells of the HepG2 cell line derived from human hepatocarcinoma, and compared with that of ICL670 in the presence and without the presence of exogenous iron. The proliferating HepG2 cell cultures were treated for 24 hours after seeding in 96-well microplates by concentrations of the different Quilamines ranging from 0.1 to 400 micromoles per liter. The cytostatic (anti-proliferative) and cytotoxic effects of Quilamine after 72 hours of treatment of the cells were assessed respectively by counting cell nuclei after DNA marking by Hoescht 33342 fluorescent intercalant or the mitochondrial succinate dehydrogenase (SDH) activity and by analysis of membrane damage detected by dosing the lactate dehydrogenase (LDH) activity in the supernatants. The results obtained on SDH activity are given in FIG. 5.

The majority of the Quilamines, like ICL670, show biphase dose-effect curves in the absence of exogenous iron, with an anti-proliferative constituent for concentration values of 1 to 10 micromoles per liter and a cytotoxic constituent associated with a release of LDH for concentration values of over 10 micromoles per liter. The percentage of HepG2 cells concerned by the anti-proliferative effect and the values of IC50 associated with this cytostatic effect in the absence of exogenous iron were listed in the table 4 below.

TABLE 3

| | % | IC₅₀ (μM) |
|---|---|---|
| HQ1-44 | 30 | 2.75 |
| HQ1-444 | 10 | 4.75 |
| HQ1-443 | 17.5 | 2.25 |
| HQ1-33 | 10 | 1.5 |
| HQ1-333 | 12.5 | 1 |
| HQ1-344 | 10 | 1.75 |
| HQ1-343 | 12.5 | 2.15 |
| HQ1-34 | 5 | 1.75 |
| HQ1-43 | 10 | 1.75 |
| ICL-670 | 72.5 | 3.85 |

Anti-proliferative effect of Quilamine compared with that of the reference chelator, deferasirox ™ (ICL670), in human hepatocarcinoma cells (HepG2).

The anti-proliferative efficacy of HQ1-44 is the highest after ICL670, followed by that of the compounds HQ1-443, HQ1-333 and HQ1-343.

In the presence of exogenous iron, an inhibition is observed of the cytostatic and cytotoxic effects induced by ICL670 while for Quilamine HQ1-44, only the cytotoxic effect is partially inhibited. As in the case of cells of the CHO cell line, this absence of inhibition of the anti-proliferative effect of Quilamines by addition of exogenous iron seems to confirm the fact that this effect is not associated solely with the intra-cell iron depletion capacity of Quilamine.

### 2. Implication of PTS in the Anti-Proliferative Action of Quilamines in Hepatocyte Tumor Cells of the HepG2 Cell Line FIG. 6)

The implication of PTS in the cytostatic and cytotoxic action of Quilamines was analyzed in proliferating HepG2 hepatocyte tumor cells after activation of PTS or competitive inhibition of PTS by spermidine, a polyamine naturally present in the organism and most efficaciously captured by the PTS. The conditions of treatment by Quilamines are identical to those described in paragraph III-1.

Inhibiting ornithine decarboxylase (ODC), the key enzyme in the biosynthesis of polyamines, causes a depletion of intra-cell polyamines and an activation of PTS which enables the deficiency to be met. This activation of PTS is done by means of the ODC inhibitor, alpha-difluoromethyl ornithine (DFMO).

The competitive inhibition of PTS by spermidine is prompted by the co-treatment of the cells by the Quilamines in the presence of spermidine, in a concentration of 50 micromoles per liter.

The results obtained with a Quilamine HQ1-44 are given in FIG. 6.

It is observed that the activation of PTS by the pre-treatment with DFMO results in an amplification of its cytotoxicity at concentrations greater than 50 micromoles per liter (50-100 micromoles per liter).

It is observed that the competitive inhibition of the capture of Quilamine by PTS results on the contrary in an inhibition of the cytostatic effects (range of concentration in Quilamine below 10 micromoles per liter) and cytotoxic effects (range of concentration in Quilamine of 50 to 100 micromoles per liter). This suggests the implication of PTS in the effects of Quilamine HQ1-44 on HepG2 hepatocyte cells.

### 3. Efficacy of Quilamines in Erocyte Tumor Cells of the Caco-2 Cell Line (FIG. 7)

The action of Quilamines on the cell viability was analyzed on enterocyte cells of the Caco-2 tumor cell line derived from human colon carcinoma and compared with the action of ICL670 in the presence or absence of exogenous iron, after activation (DFMO) or competitive inhibition by spermidine, of the PTS. The operating conditions used are identical to those used for CHO/CHO-MG and HepG2 cells.

As in the case of the other cell lines in proliferative phase presented here above, the dose-effect curves of the action of the Quilamines on the number of viable cells (number of nuclei) are biphase curves with a cytostatic constituent for concentration values of below 30 micromoles per liter and a cytotoxic constituent with release of LDH for concentration values above 30 micromoles per liter. Only this second cytotoxic constituent is observed when the cells of the Caco-2 cell line are treated after confluence (stopping of the proliferation).

The results for the measurement of the anti-proliferative effect of Quilamines, and of ICL670 on the proliferating cells (cytostatic constituent) when there is no exogenous iron have been listed in the table 4 below. The 8-hydroxyquinoline (8-HQ) has only one cytotoxic constituent with an IC50 of 5 micromoles per liter.

TABLE 4
Anti-proliferative effect of Quilamines compared
with that of the reference chelators, 8-hydroxyquinoline
(8-HQ) and deferasirox ™ (ICL670), in human
colon adenocarcinoma cells (Caco-2).

| | Constituent 1 | | Constituent 2 | | | |
|---|---|---|---|---|---|---|
| | (1-10 μM) | | (10-100 μM) | | % LDH/control | |
| Caco-2 J4 | % cell | IC50 (μM) | % cell | IC50 (μM) | 10 μM | 100 μM |
| HQ1-44 | 70 | 1.3 | 30 | 20 | 118 | 287 |
| HQ1-34 | 20 | 1 | 80 | 45 | 100 | 229 |
| HQ1-43 | 7 | 1 | 93 | 30 | 93 | 240 |
| HQ1-33 | 40 | 0.8 | 60 | 37 | 118 | 261 |
| HQ1-444 | 60 | 2 | 40 | 75 | 99 | 118 |
| HQ1-443 | 5 | 1 | 95 | 35 | 88 | 189 |
| HQ1-344 | 22 | 3 | 78 | 75 | 95 | 120 |
| HQ1-343 | 30 | 2 | 70 | 40 | 89 | 255 |
| HQ1-333 | 20 | 3 | 80 | 60 | 112 | 169 |
| 8-HQ | — | — | 100 | 5 | 281 | 353 |
| ICL670 | 18 | 1.7 | 82 | 90 | 117 | 130 |

After addition of exogenous iron, an inhibition is observed of the cytostatic and cytotoxic effects induced by ICL670 while, for Quilamine HQ1-44, only the cytotoxic effect is partially inhibited. This absence of inhibition of the anti-proliferative effect of Quilamines by addition of exogenous iron seems to confirm that this effect is not associated only with the intra-cell iron depletion capacity of Quilamine.

### 3. Anti-Tumor Action of Quilamines by Intra-Cell Iron Depletion in Enterocyte Tumor Cells of the Caco-2 Cell Line

After treatment for 72 hours of the Caco-2 cells in proliferative phase by the Quilamine HQ1-44 and ICL670 in a concentration of 10 micromoles per liter, an analysis is carried out by real-time polymerase chain reaction (RT-qPCR) technique of the expression of the two main genes for regulating intra-cell iron metabolism, that of L-ferritin, the iron-storing protein, and that of the transferrin receptor, which is the iron-importing protein of the membrane. The results are shown in FIG. 8. The DNAc (complementary dioxyribonucleic acid) synthesized during the reverse transcription are amplified by the qPCR (quantity of polymerization chain reaction) to quantify the expression of the mNRA (messenger ribonucleic acid) of the genes involved in iron metabolism.

After treatment for 72 hours of the Caco-2 cells in proliferative phase by Quilamine HQ1-44 and ICL670, biochemical analysis is carried out on the intra-cell iron (Fe2+ and Fe3+), of the two main proteins for regulating iron metabolism by the dosing of the intra-cell concentrations in L-ferritin, the iron storage protein, as well as the analysis of the transferrin soluble receptor the cell supernatant, which is a marker of the concentration in transferrin receptor, the iron importing protein of the membrane. The results are presented in FIG. 9.

On the results of the Caco-2 cells in proliferative phase, the Quilamine HQ1-44 and ICL670 in a concentration of 10 micromoles per liter, do not significantly modify the expression of the encoding genes for the iron storage protein, L-ferritine (L-Iron), and for the main path of entry of iron into the cell, the transferrin receptor (RTrf). The exposure of the cells to iron-saturated transferrin (holotransferrin) does not modify the expression of the L-ferritin and reduces the expression of the transferrin receptor. The iron overload induced by the exposure for 72 hours at 20 micromoles per liter of iron-citrate is accompanied by a small (non-significant) increase in the expression of L-ferritin and an inhibition of the expression of the transferrin receptor, according to the IRE/IRP regulation of these two genes.

The biochemical analysis of the concentrations in L-ferritin and in iron show that Quilamine HQ1-44 and ICL670 in a concentration of 10 micromoles per liter cause intra-cell iron depletion. The treatment by these chelators has no significant effect on the transferrin-soluble receptor. Conversely, exposure to holotransferrin (iron-saturated Trf) and iron-citrate is accompanied by a marked increase in L-ferritin and intra-cell iron.

### 5. Anti-Tumor Action of Quilamines by Implication of Polyamine Metabolism in the Enterocyte Tumor Cells of the Caco-2 Cell Line

After treatment for 72 hours of the Caco-2 cells, in proliferative phase by Quilamine HQ1-44 and ICL670 in a concentration of 10 micromoles per liter, an analysis is carried by RT-qPCR of the expression of the main genes for regulating the metabolism of polyamines, such as ornithine decarboxylase (ODC), antizyme (OAZ1), S-adenosyl methionine decarboxylase (SAMDC) and polyamine oxydase (PAO). The results are shown in FIG. 10.

After treatment for 72 hours of the Caco-2 cells, in proliferative phase by Quilamine HQ1-44 and ICL670 in a concentration of 10 micromoles per liter, a biochemical analysis is carried out by tandem mass spectrometry coupled with liquid chromatography (LC/MSMS) of the intra-cell polyamines such as putrescine, spermidine and spermine, after dansylation. The results are presented in FIG. 11.

Treatment for 72 hours of the proliferating Caco-2 cells by HQ1-44 chelators and ICL670 chelators in a concentration of 10 micromoles per liter has no effect on the expression of the genes implicated in the regulation of the metabolism of the polyamine like that of ODC, the antizyme (OAZ1), S-adenosyl methionine decarboxylase (SAMDC) and that of the polyamine oxydase (PAO). Ctrate alone prompts the reduction of the expression of genes involved in the biosynthesis of the polyamines (ODC and SAMDC). By contrast, the iron overload caused by holotransferrin or iron-citrate has no effect on the expression of the genes of the metabolism of the polyamines.

The iron overload does not modify the concentrations of natural polyamines (putrescine, spermidine and spermine). By contrast, the intra-cell concentrations of putrescine (Put) and of spermidine (Spd) are lowered after treatment by Quilamine HQ1-44 (−73% Put, −55% Spd) and to a lesser extent by ICL670 (−34% Put, −15% Spd). This reduction of the Put, associated with a more limited diminishing of the Spd and the absence of an effect on intra-cell Spm is comparable to the effect of DFMO, the ODC inhibitor.

This reduction of the intra-cell polyamines, putrescine and spermidine, induced in the proliferating Caco-2 cells by Quilamine HQ1-44 and to a lesser degree with ICL670 in a concentration of 10 micromoles per liter, contributes to the anti-proliferative effect of these chelators jointly with the iron depletion that they cause. The influence of the inhibition of intra-cell polyamines by HQ1-44 on cell proliferation would explain the absence of inhibition of the anti-proliferative effect of Quilamines by exogenous iron.

1. Compound characterized in that it responds to the following general formula (I) or to one of its conjugate forms, its salts or its solvates: 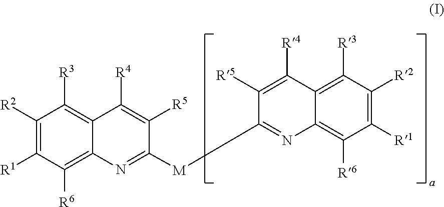
wherein:
R¹, R², R³, R⁴ and R⁵ are chosen independently of one another from among a hydrogen atom, a linear or branched, saturated and non-saturated, cyclic or aromatic hydrocarbon group comprising 1 to 10 carbon atoms, an aliphatic or aromatic cyclic hydrocarbon group comprising 4 to 12 carbon atoms, a halogen, a thiol, a hydroxyl, an amine that is preferably a secondary or tertiary amine, an ether, a thioether;
R⁶ is a hydroxyl;
a is equal to 0 or 1 with the following:
when a is equal to 0, M is a group responding to the following formula (I′): 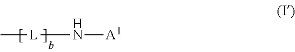
wherein:
b being equal to 0 or 1,
L representing an alkyl group having 1 to 4 carbon atoms, a group C═O or C═S,
A¹ responding to the general formula (X) or (Y): 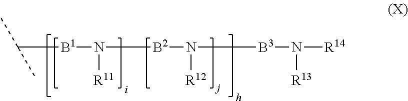
wherein:
R¹¹, R¹², R¹³ and R¹⁴ are chosen independently of one another from among hydrogen atoms, linear or branched, saturated, unsaturated, cyclic or aromatic hydrocarbon groups comprising 1 to 12 carbon atoms, and amine function protecting groups,
B¹, B² and B³ are chosen independently of one another from among linear or branched, saturated or unsaturated, cyclic or aromatic hydrocarbon groups comprising 2 to 6 carbon atoms
i and j, identical or different are equal to 0 or to 1,
h is an integer ranging from 0 to 4; 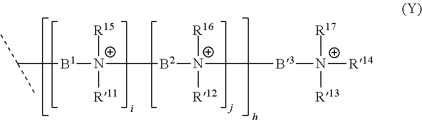
wherein:
R′¹¹, R′¹², R′¹³, R′¹⁴, R¹⁵, R¹⁶, and R¹⁷ are chosen independently of one another from among the linear or branched, saturated, unsaturated, cyclic or aromatic hydrocarbon groups comprising 1 to 12 carbon atoms and amine function protecting groups.
B^{1 and} B² are chosen independently of one another from among the linear or branched, saturated or unsaturated hydrocarbon groups comprising 2 to 6 carbon atoms,
B′³ is chosen from the linear or branched or saturated, unsaturated hydrocarbon groups comprising 2 to 6 carbon atoms capable of being interrupted by one or more secondary amine functions —NH— and/or one or more tertiary amine functions —NR¹¹—, —NR¹²—.
i and j, which are identical or different, are equal to 0 or to 1,
h is an integer ranging from 0 to 4;
and when a is equal to 1, M is a group meeting the following formula (I″): 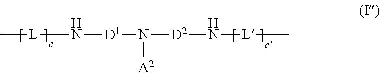
wherein:
c and c′, identical or different, being equal to 0 or to 1,
L and L′, identical or different, represent an alkyl group having to 1 to 4 carbon atoms, a group C═O or C═S,
D^{1 and} D², identical or different, represent a linear or branched hydrocarbon group, comprising 1 to 5 carbon atoms,
A² being identical or different from A¹ and meeting the formula (X) or (Y);
R′¹, R′², R′³, R′⁴, R′⁵ and R′⁶ are chosen independently of one another from among a hydrogen atom, a linear or branched, saturated or unsaturated hydrocarbon group comprising 1 to 10 carbon atoms, a cyclic, aliphatic or aromatic, possibly substituted hydrocarbon group comprising 4 to 12 carbon atoms, a halogen, a thiol, hydroxyl, a preferably secondary or tertiary amine, an ether, a thioether.

2. Compound according to claim 1, characterized in that it is a metal chelator.

3. Compound according to claim 2, characterized in that it is a chelator of iron, copper, manganese and/or zinc.

4. Compound according to claim 1, characterized in that the polyamine chain or chains are cationic or cationizable.

5. Compound according to claim 1, characterized in that a is equal to 1, c and c′ are identical, L and L′ are identical, D¹ and D² are identical.

6. Compound according to claim 1, characterized in that R¹, R², R³, R⁴, R⁵ and R⁶ are identical to R′¹, R′², R′³, R′⁴, R′⁵ and R′⁶ respectively.

7. Compound according to claim 1, characterized in that R′⁶ is a hydroxyl.

8. Compound according to the preceding claim, characterized in that B¹, B², B³ are chosen from the groups n-propyl and n-butyl and B′³ is chosen from the groups n-propyl and n-butyl capable of being interrupted by one or more secondary amine functions —NH— and/or one or more tertiary amine functions —NR¹¹—, —NR¹²—.

9. Compound according to claim 1, characterized in that it responds to the formula (I) in which R¹ to R⁵ are chosen independently of one another from among a hydrogen atom, a linear or branched, saturated or unsaturated hydrocarbon group comprising 1 to 10 carbon atoms; R⁶ is a hydroxyl; a is equal to 0; b is equal to 1; L is an alkyl group in C₁-C₂; and the group A¹ responds to the formula (X) in which R¹¹, R¹², R¹³ and R¹⁴ are chosen independently of one another from among hydrogen atoms and amine function protecting groups; B¹, B² and B³ which are identical or different, being n-propyl and/or n-butyl groups; i and j which are identical or different being equal to 1 or to 0; h being equal to 1.

10. Compound according to claim 1, as a therapeutic agent for humans and animals.

11. Compound according to claim 10, as an agent for treating proliferative diseases,

12. Compound according to claim 10, as an agent for treating neurodegenerative diseases.

13. Compound according to claim 10 diseases related to an overload of iron, copper, zinc and/or manganese, in human or animal cells.

14. Compound according to claim 10, as an agent inhibiting the synthesis of polyamines by the organism.

15. Composition comprising at least one compound as defined in claim 1 and possibly at least one additional active principle, said compound or compounds and active principle or principles, if any, of the composition being capable of being used simultaneously, separately or in a manner that is spread over time.

16. Composition according to claim 15, characterized in that the additional active principle is chosen from among agents inhibiting the endogenous synthesis of polyamines and chemotherapeutic agents.
